(19) United States
(12) Patent Application Publication
(10) Pub. No.: US 2010/0144848 A1
(43) Pub. Date: Jun. 10, 2010

(54) DNA CONTROLLED ASSEMBLY OF LIPID MEMBRANES

(75) Inventors: Stefan Vogel, Odense So (DK); Adam Cohen Simonsen, Odense C (DK)

Correspondence Address: KNOBBE MARTENS OLSON & BEAR LLP, 2040 MAIN STREET, FOURTEENTH FLOOR, IRVINE, CA, 92614, US

(73) Assignee: SYDDANSK UNIVERSITET, Odense M (DK)

(21) Appl. No.: 12/524,232

(22) PCT Filed: Jan. 24, 2008
(86) PCT No.: PCT/DK2008/050011

§ 371 Date: Feb. 17, 2010

(30) Foreign Application Priority Data
Jan. 24, 2007 (DK) PA 2007 00110

(51) Int. Cl. A61K 31/711; C07H 21/02; C40B 40/06; G01N 33/48; C07H 21/04; A61K 31/7105; A61P 35/00

(52) U.S. Cl. 514/44 R; 536/23.1; 506/16; 436/94

(57) ABSTRACT

The present invention relates to detection of target nucleic acids by target nucleic acid induced liposome assembly. The invention provides oligonucleotides for use in detection and a method of detecting target nucleic acids. Other aspects of the invention are use of the oligonucleotide of the invention for detection, a kit for detection, a method of treatment comprising administrating the oligonucleotide of the invention and a pharmaceutical composition comprising the oligonucleotide of the invention.

### FIELD OF THE INVENTION

The invention relates to oligonucleotides and their use in detection and therapy.

### BACKGROUND

The further development of methods for sequence specific detection of nucleic acids is of outermost importance in various fields of life science. In particular such methods are used in relation to genetic diseases and for identification of disease causing pathogens. Also in the further development of personalized medicine, such methods are of relevance.

Very often PCR (polymerase chain reaction) is used for sequence specific detection of nucleic acids. The function of PCR may be to increase the concentration of the target nucleic acid before sequence specific detection of particular genetic markers, and the PCR may itself also be used for detection of particular markers. Detection may also be based on hybridisation of probes that are fluorescently labelled.

Both way, PCR and related detection techniques require sophisticated machinery and often are more time consuming than desirable.

There is clearly a need for improved methods of detecting nucleic acids, and of particular interest are more simple and low cost methods with improved sensitivity.

In addition to being used in relation to detection, oligonucleotides may also be used as therapeutics compounds. Examples are antisense oligonucleotides, siRNAs, microRNAs, ribozymes etc. The compounds all have the promise of being able to sequence specifically target a cellular nucleic acid, typically an mRNA.

A draw back of therapeutic oligonucleotides is their poor delivery into cells, due to their polyanionic nature. Therefore, delivery is often sought mediated by lipids, e.g. in the form of liposomes or by conjugation of a lipid to the oligonucleotide. Still, drawbacks exist. Thus, an oligonucleotide to which a lipid has been conjugated will have non-desirable characteristics because the lipid will give the oligonucleotide a tendency to aggregate, i.e. the lipids of different oligonucleotides will aggregate because of their hydrophobicity.

### SUMMARY OF THE INVENTION

The present invention relates to an amphiphilic oligonucleotide comprising two membrane anchors and methods for using the aforementioned oligonucleotide. In a first aspect, the invention provides a method for constructing oligonucleotide-lipid membrane architectures. Said oligonucleotide-lipid membrane architectures are useful for detection and quantification of a target nucleic acid. The oligonucleotide-lipid membrane architecture can also be used for separation of a target nucleic acid from a mixture. Another aspect of the invention is the oligonucleotide comprising two membrane anchors for use in medicine.

### BRIEF DESCRIPTION OF DRAWINGS

FIG. 1. General Chemical core structure of a membrane anchor exemplified as modification X.

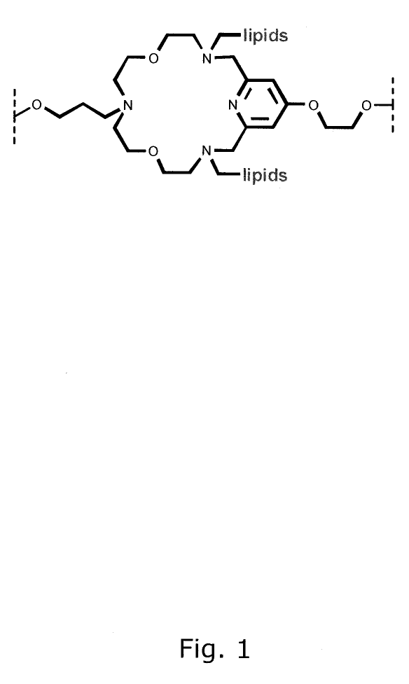

FIG. 2. Table of exemplified sequences.

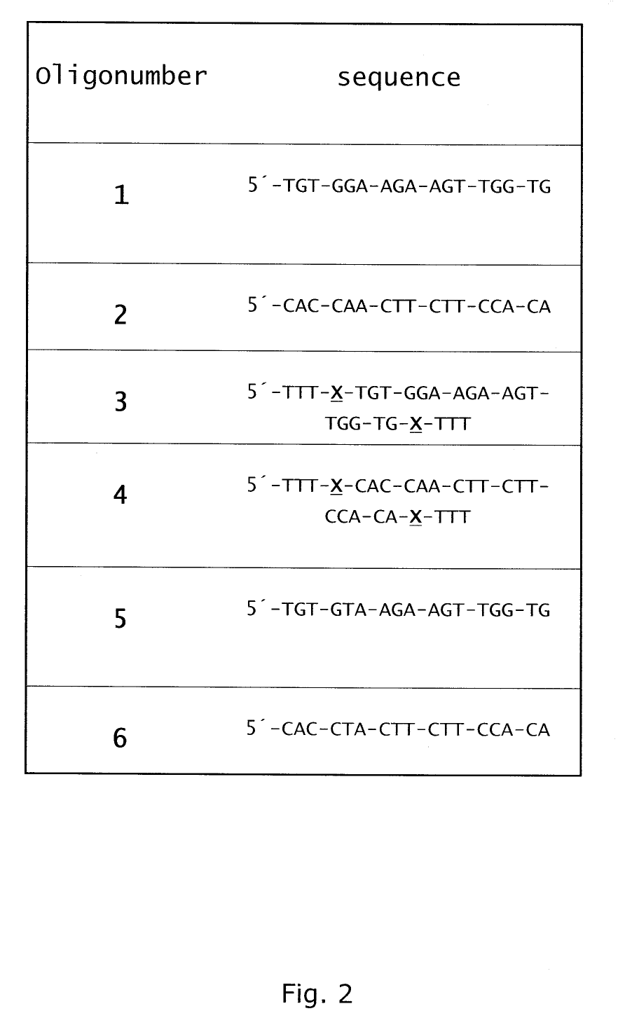

FIG. 3. Mode of action for the interaction of sequences (ID 3 and ID 4) with lipid bilayers (exemplified using vesicles in the size of 30, 50, 100, 200 and 400 nm).

FIG. 4. Mode of action for the interaction of sequences (ID 3 and ID 4) with lipid bilayers (exemplified using vesicles in the size of 30, 50, 100, 200 and 400 nm) when hybridised to a target nucleic acid as exemplified for DNA (sequence ID 1, ID2, ID 5 and ID 6)

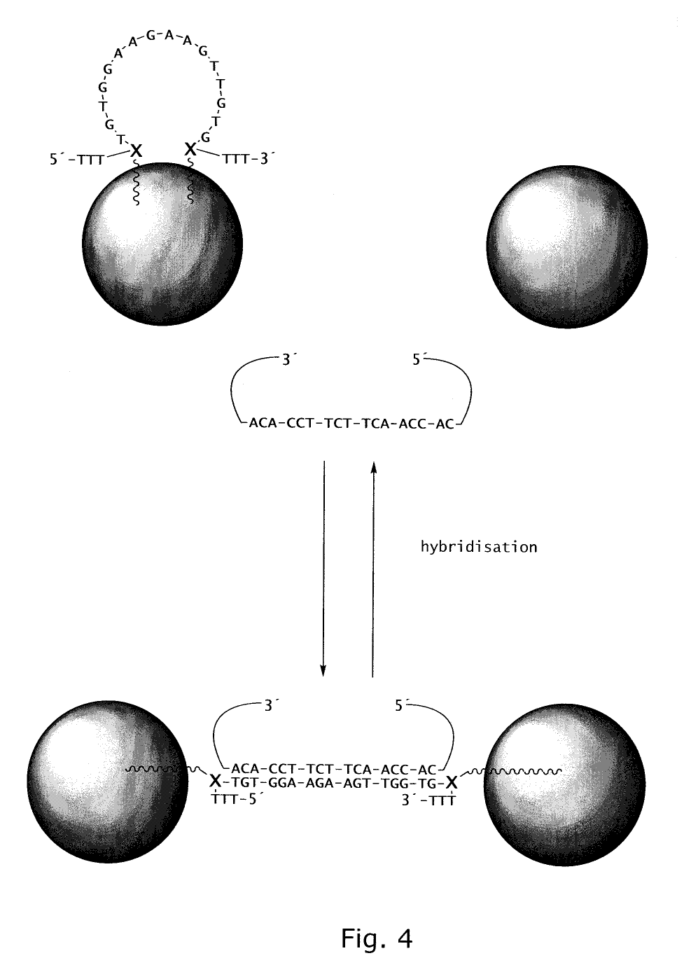

FIG. 5. Mode of action for the interaction of sequences (ID 3 and ID 4) with lipid bilayers (exemplified using vesicles in the size of 30, 50, 100, 200 and 400 nm) when hybridised to a target nucleic acid as exemplified for DNA (sequence ID 1, ID2, ID 5 and ID 6) and the mode of action for the lipid bilayer assembly process as result of the hybridisation of the probe sequence (sequence ID 3 and ID 4) with any of the exemplified target sequence (sequence ID 1, ID2, ID 5 and ID 6)

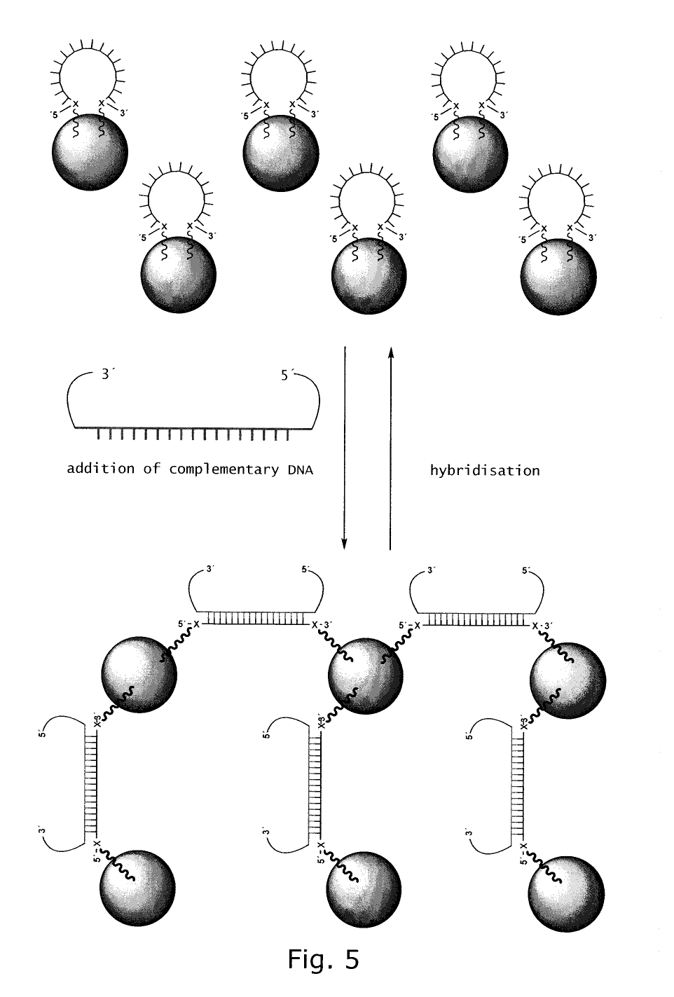

FIG. 6. Thermal denaturation curves recorded in a UV experiment at 260 nm wavelength showing hybridisation of sequence ID1 and ID2 compared to the hybridisation of sequence ID3 and ID4

FIG. 7. First derivative of the thermal denaturation curves recorded in a UV experiment at 260 nm wavelength showing hybridisation of sequence ID1 and ID2 compared to the hybridisation of sequence ID3 and ID4

FIG. 8. Thermal denaturation curves recorded in a UV experiment at 260 nm wavelength showing hybridisation of sequence ID1 and ID2 compared to the hybridisation of sequence ID3 and ID4 and run in two consecutive cycles from 15° C. to 80° C. showing the reproducibility of the assembly and disassembly process of the bilayers used (vesicles).

FIG. 9. First derivative of the thermal denaturation curves recorded in a UV experiment at 260 nm wavelength showing hybridisation of sequence ID1 and ID2 compared to the hybridisation of sequence ID3 and ID4 and run in two consecutive cycles from 15° C. to 80° C. showing the reproducibility of the assembly and disassembly process of the bilayers used (vesicles).

FIG. 10. Thermal denaturation curves recorded in a UV experiment at 260 nm wavelength showing hybridisation of sequence ID4 and ID1 compared to the hybridisation of sequence ID4 and ID5 at 0.125 μM DNA concentration of each single stranded DNA sequence

FIG. 11. Thermal denaturation curves recorded in a UV experiment at 260 nm wavelength showing hybridisation of sequence ID3 and ID2 compared to the hybridisation of sequence ID3 and ID6 at 0.125 μM DNA concentration of each single stranded DNA sequence

FIG. 12. Thermal denaturation curves recorded in a UV experiment at 260 nm wavelength showing hybridisation of sequence ID4 and ID1 compared to the hybridisation of sequence ID4 and ID5 at 12.5 nM DNA concentration of each single stranded DNA sequence.

FIG. 13. Thermal denaturation curves recorded in a UV experiment at 260 nm wavelength showing hybridisation of sequence ID3 and ID2 compared to the hybridisation of sequence ID3 and ID6 at 0.125 μM DNA concentration of each single stranded DNA sequence

FIG. 14. Synthesis of macrocyclic building blocks as membrane anchor units in DNA-conjugates (sequence ID 3 and sequence ID 4)

FIG. 15. Synthesis of a building block for the synthesis of macrocycles in FIG. 14 as membrane anchor units in DNA-conjugates (sequence ID 3 and sequence ID 4)

FIG. 16. Thermal denaturation curves for testing target oligonucleotide mediated liposome assembly. See example 8.

FIG. 17. Thermal denaturation curves for mismatch discrimination studies. See example 9.

FIG. 18. Thermal denaturation curves of an Anthrax sequence. See example 10.

FIG. 19. Thermal denaturation curves of Staphylococcus aureus sequences. See example 11.

FIG. 20. Thermal denaturation curves of a Staphylococcus aureus sequence. See example 12.

FIG. 21. 3-strand design for liposome assembly. See example 13.

FIG. 22. Thermal denaturation curves for 3-strand design. See example 13.

FIG. 23. Immobilisation of oligonucleotides and liposomes on a solid support. See example 14.

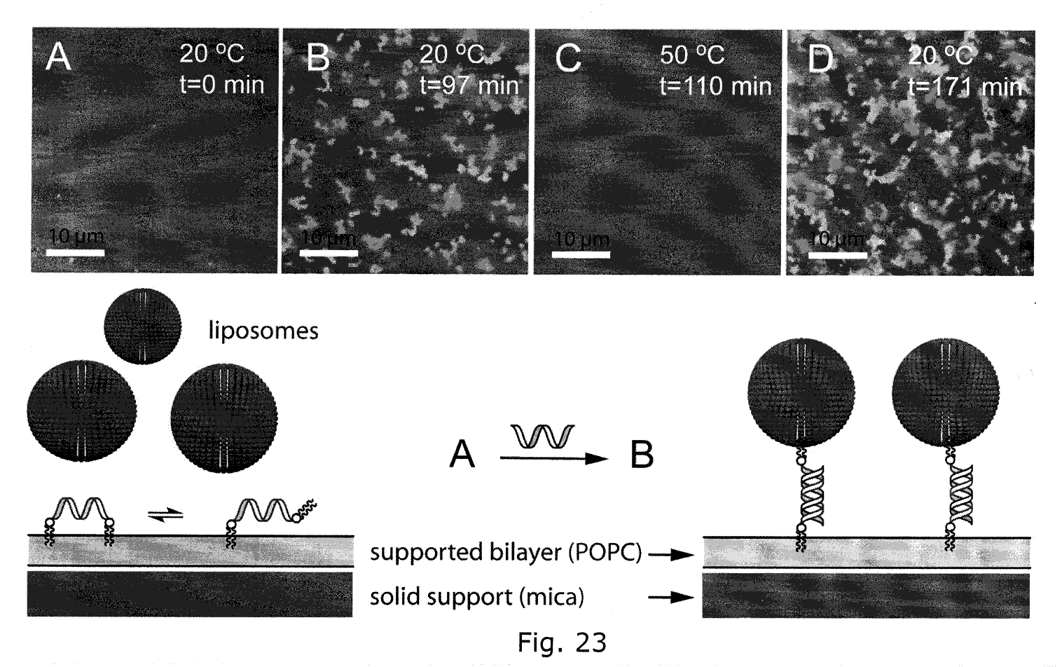

### DETAILED DESCRIPTION OF THE INVENTION

### Method for Constructing Oligonucleotide-Lipid Architectures

In a first aspect, the present invention provides a method for constructing oligonucleotide-lipid architectures comprising the steps of:
a) Providing one or more amphiphilic oligonucleotides, together comprising at least two membrane anchors
b) Providing a lipid membrane
c) Incubating the amphiphilic oligonucleotide with the lipid membrane, under conditions allowing insertion of the membrane anchors into the lipid membrane.

When performing steps a-c, the membrane anchors of the one or more amphiphilic oligonucleotides will insert into the lipid membrane. The resulting oligonucleotide-lipid membrane architecture has a number of applications, as will be clear from the embodiments described below.

In a preferred embodiment of the first aspect of the invention, the method further comprises incubating, under conditions of hybridisation, the one or more amphiphilic oligonucleotides and the lipid membrane with a target nucleic acid that comprises a region of complementarity to the one or more amphiphilic oligonucleotides. The effect of doing so is target nucleic acid controlled lipid membrane aggregation, i.e. the bringing together of lipid membranes. Thus, hybridisation of the amphiphilic oligonucleotide to the target nucleic acid can be detected by detecting lipid membrane aggregation. A major advantage of this mechanism is that lipid membrane aggregation can, at certain concentrations of lipid membranes, amphiphilic oligonucleotides and target nucleic acids, actually be detected by visual inspection of the sample, as the sample will turn milky. Thus, hybridisation and ultimately, the presence of the target nucleic acid can be detected by visual inspection, without sophisticated equipment.

Conditions of hybridisation are well-known to the skilled man. Adjustable parameters include e.g. temperature and ionic strength.

When referring to a “region of complementarity”, what is meant is a region that can form a duplex with the amphiphilic oligonucleotide through base pairing. Preferred base pairs are G:C, C:G, A:T, T:A, A:U, U:A, G:U and U:G. In some embodiments, universal bases that allow base pairing to all natural occurring bases are included in either the amphiphilic oligonucleotide or the target nucleic acid.

When more than one amphiphilic oligonucleotide is employed, they are preferably capable of forming a contiguous double stranded complex with the region of complementarity of the target nucleic acid. In some embodiments, a gap of 1, 2, 3 or 4 nucleotides may be present between the amphiphilic oligonucleotides, when base paired to the target nucleic acid. In another embodiment, two amphiphilic oligonucleotides may be separated by one or more oligonucleotides that do not comprise a membrane anchor. The multi-oligonucleotide structures will all prevent the two membrane anchors from inserting into the same lipid membrane, wherefore they will promote lipid assembly.

### The Amphiphilic Oligonucleotide

In a preferred embodiment, the method only employs one amphiphilic oligonucleotide. As will be apparent, many of the embodiments described below, which is related to only one amphiphilic oligonucleotide will also be useful for embodiments that employs two or more amphiphilic oligonucleotides.

Preferably, the amphiphilic oligonucleotide comprises only 2 membrane anchors.

The two anchors of the amphiphilic oligonucleotide should be separated by at least 5 nucleotides. Otherwise, there may be steric problems in the insertion of both anchors into the lipid membrane. Thus, in a preferred embodiment, two anchors of the amphiphilic oligonucleotide are separated by a distance selected from the group consisting of at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least, 25 nucleotides, at least 26 nucleotides, at least 27 nucleotides, at least 28 nucleotides, at least 29 nucleotides and at least 30 nucleotides.

In another preferred embodiment, two anchors of the amphiphilic oligonucleotide are separated by a distance selected from the group consisting of less than 6 nucleotides, less than 7 nucleotides, less than 8 nucleotides, less than 9 nucleotides, less than 10 nucleotides, less than 11 nucleotides, less than 12 nucleotides, less than 13 nucleotides, less than 14 nucleotides, less than 15 nucleotides, less than 16 nucleotides, less than 17 nucleotides, less than 18 nucleotides, less than 19 nucleotides, less than 20 nucleotides, less than 21 nucleotides, less than 22 nucleotides, less than 23 nucleotides, less than 24 nucleotides, less than, 25 nucleotides, less than 26 nucleotides, less than 27 nucleotides, less than 28, less than 29 nucleotides, less than 30 nucleotides, less than 40 nucleotides and less than 50 nucleotides.

The amphiphilic oligonucleotide may comprise monomers selected from the group consisting of DNA monomers, RNA monomers, LNA monomers, PNA monomers, INA monomers and morpholino monomers. LNA, PNA, INA and morpholino monomers may be used to increase the melting temperature of the amphiphilic oligonucleotide hybridised to a complementary nucleic acid. Other nucleotide monomers may also be present in the amphiphilic oligonucleotide.

In a preferred embodiment, the length of the amphiphilic oligonucleotide is less than 35 nucleotides. In another embodiment, the length of the amphiphilic oligonucleotide is selected from the group consisting of less than 6 nucleotides, less than 7 nucleotides, less than 8 nucleotides, less than 9 nucleotides, less than 10 nucleotides, less than 11 nucleotides, less than 12 nucleotides, less than 13 nucleotides, less than 14 nucleotides, less than 15 nucleotides, less than 16 nucleotides, less than 17 nucleotides, less than 18 nucleotides, less than 19 nucleotides, less than 20 nucleotides, less than 21 nucleotides, less than 22 nucleotides, less than 23 nucleotides, less than 24 nucleotides, less than, 25 nucleotides, less than 26 nucleotides, less than 27 nucleotides, less than 28, less than 29 nucleotides, less than 30 nucleotides, less than 40 nucleotides and less than 50 nucleotides.

In yet another preferred embodiment, the length of the amphiphilic oligonucleotide is selected from the group consisting of at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least, 25 nucleotides, at least 26 nucleotides, at least 27 nucleotides, at least 28 nucleotides, at least 29 nucleotides and at least 30 nucleotides.

The length of the region of complementarity can be adjusted e.g. for optimization of the melting temperature of the amphiphilic oligonucleotide hybridised to the target nucleic acid. Thus, in preferred embodiment, the region of complementarity has a length selected from the group consisting less than 6 nucleotides, less than 7 nucleotides, less than 8 nucleotides, less than 9 nucleotides, less than 10 nucleotides, less than 11 nucleotides, less than 12 nucleotides, less than 13 nucleotides, less than 14 nucleotides, less than 15 nucleotides, less than 16 nucleotides, less than 17 nucleotides, less than 18 nucleotides, less than 19 nucleotides, less than 20 nucleotides, less than 21 nucleotides, less than 22 nucleotides, less than 23 nucleotides, less than 24 nucleotides, less than, 25 nucleotides, less than 26 nucleotides, less than 27 nucleotides, less than 28, less than 29 nucleotides, less than 30 nucleotides, less than 40 nucleotides and less than 50 nucleotides.

In yet another embodiment, the region of complementarity has a length selected from the group consisting of at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least, 25 nucleotides, at least 26 nucleotides, at least 27 nucleotides, at least 28 nucleotides, at least 29 nucleotides and at least 30 nucleotides. As mentioned above the length of the region of complementarity as well as nucleotide analogues can be used to adjust the melting temperature of the amphiphilic oligonucleotide hybridised to the target nucleic acid.

In a preferred embodiment, the melting temperature is within a range of 5° C. of the temperature in which the method is performed. Even more preferred is a range of 3° C. or 2° C. By using such a melting temperature, specificity is assured and only perfect hybridisation with no mismatches will occur.

As will be clear from the examples section, the method of the invention gives a very steep (or sharp) melting curve of the amphiphilic oligonucleotide hybridised to a target nucleic acid in the presence of a lipid membrane. Not intended to be bound by theory, it is believed that the steep melting curve is caused by double cooperativity introduced by the presence of the lipid membrane. It is one object of the invention to use this improved discrimination between a perfect duplex and a duplex with one or more mismatches e.g. for more specific detection and quantification of a target nucleic acid.

In a preferred embodiment, the region of complementarity hybridised to a complementary RNA oligonucleotide has a melting temperature of at least 30° C., such as at least 40° C., such as at least 50° C., such as at least 60° C.

### Membrane Anchors

As described above, the membrane anchors should be capable of insertion into a lipid membrane. Thus, membrane anchors are defined herein as moieties build into an oligonucleotide with the purpose of allowing attachment of the oligonucleotide to a lipid membrane.

In a preferred embodiment, membrane anchors are inserted as monomer building blocks during oligonucleotide synthesis. Thus, membrane anchors may comprise any lipophilic group that will allow insertion into a lipid membrane. Such lipophilic groups are known to the skilled man and may e.g. be fatty acids, steroids etc.

In a preferred embodiment, at least 1 membrane anchor of the amphiphilic oligonucleotide inserts reversibly into the lipid membrane, such that the particular membrane anchor may change position from one lipid membrane to another lipid membrane.

Reversible insertion into a lipid membrane is simple to achieve and demands that the lipohilic group of the membrane anchor does not comprise lipophilic groups of excessive size.

A simple method to test for reversibility is based on aggregation of lipid membranes, as outlined in the examples section. If not at least one membrane anchor inserts reversibly into the lipid membrane, lipid membrane aggregation cannot be achieved, which is easily detected.

In a preferred embodiment, at least one membrane anchor comprise an alkyl chain with a length selected from the group consisting of: at least 4 C atoms, at least 5 C-atoms, at least 6 C-atoms, at least 7 C-atoms, at least 8 C-atoms, at least 9 C-atoms, at least 10 C-atoms, at least 11 C-atoms, at least 12 C-atoms, at least 13 C-atoms, at least 14 C-atoms, at least 15 C-atoms, at least 16 C-atoms, at least 17 C-atoms, at least 18 C-atoms, at least 19 C-atoms and at least 20 C-atoms, an alkenyl chain with a length selected from the group consisting of: at least 4 C atoms, at least 5 C-atoms, at least 6 C-atoms, at least 7 C-atoms, at least 8 C-atoms, at least 9 C-atoms, at least 10 C-atoms, at least 11 C-atoms, at least 12 C-atoms, at least 13 C-atoms, at least 14 C-atoms, at least 15 C-atoms, at least 16 C-atoms, at least 17 C-atoms, at least 18 C-atoms, at least 19 C-atoms and at least 20 C-atoms, an alkynyl chain with a length selected from the group consisting of: at least 4 C atoms, at least 5 C-atoms, at least 6 C-atoms, at least 7 C-atoms, at least 8 C-atoms, at least 9 C-atoms, at least 10 C-atoms, at least 11 C-atoms, at least 12 C-atoms, at least 13 C-atoms, at least 14 C-atoms, at least 15 C-atoms, at least 16 C-atoms, at least 17 C-atoms, at least 18 C-atoms, at least 19 C-atoms and at least 20 C-atoms.

In another preferred embodiment, at least one membrane anchor comprise one or more aromatic rings. The aromatic rings may be heteroaromatic rings.

In yet another embodiment, at least one membrane anchor comprise a fatty acid selected from the group consisting of: butanoic acid; hexanoic acid; octanoic acid; decanoic acid; dodecanoic acid; tetradecanoic acid; hexadecanoic acid; 9-hexadecenoic acid; octadecanoic acid; 9-octadecenoic acid; 11-octadecenoic acid; 9,12-octadecadienoic acid; 9,12,15-octadecatrienoic acid; 6,9,12-octadecatrienoic acid; eicosanoic acid; 9-eicosenoic acid; 5,8,11,14-eicosatetraenoic acid; 5,8,11,14,17-eicosapentaenoic acid; docosanoic acid; 13-docosenoic acid; 4,7,10,13,16,19-docosahexaenoic acid and tetracosanoic acid.

Preferably, each membrane anchor comprise lipophilic groups selected from the groups mentioned above, e.g. two fatty acids. If only one lipophilic group is present, the membrane anchor may not be strong enough, i.e. the membrane anchor will not have a sufficient tendency to insert into the membrane. Thus, membrane anchors should neither be too strong, nor too weak.

When two lipophilic groups are present in the membrane anchor, they should preferably be positioned such as to not engage in stable hydrophobic interactions with each other. Thus, preferably, they are separated by a number of bonds selected from at least 2 bonds, at least 3 bonds, at least 4 bonds and at least 5 bonds. Preferably, the bonds are conformationally restrained.

A preferred scaffold for the membrane anchor is polyaza crown ether.

Thus, in a preferred embodiment, at least one membrane anchor comprise a polyaza crown ether with two lipophilic substituents.

Membrane anchors may be located at the 3′ terminal nucleotide and at the 5′ terminal nucleotide of the amphiphilic oligonucleotide.

In another embodiment, a first membrane anchor is located at least 1 nucleotide from the 5′ end of the amphiphilic oligonucleotide and wherein a second membrane anchor is located at least 1 nucleotides from the 3′ end of the amphiphilic oligonucleotide.

In still another embodiment, the distance between the first membrane anchor and the 5′ end is selected from the group consisting of at least 2 nucleotides, at least 3 nucleotides, at least 4 nucleotides, at least 5 nucleotides and least 6 nucleotides.

In still another embodiment, the distance between the second membrane anchor and the 3′ end is selected from the group consisting of at least 2 nucleotides, at least 3 nucleotides, at least 4 nucleotides, at least 5 nucleotides and least 6 nucleotides.

It may be desirable to have a distance between the terminal ends of the amphiphilic oligonucleotide and the membrane anchors to introduce steric hindrance that will prevent the membrane anchors or the lipophilic moieties of the membrane anchors from interacting. In particular, it is preferred that two lipophilic moieties of the same amphiphilic oligonucleotide are prevented from interaction, which can be obtained by having a distance between the terminal ends of the oligonucleotide and the membrane anchors.

### Lipid Membranes

The lipid membranes of the invention may be mono-layered membranes or bi-layered membranes.

Further, the lipid may be part of a vesicle, a micelle, a cell membrane or a cellular membrane.

Phospholipid vesicles may be prepared as outlined in the examples section.

Preferably, the size of the vesicle or micelle selected from the group consisting of: more than 20 nm, more than 50 nm, more than 75 nm, more than 100 nm, more than 150 nm and more than 200 nm.

More preferably, the size of the vesicle or micelle is selected from the group consisting of between 20 and 300 nm, between 50 and 200 nm and between 50 and 100 nm. The size of the prepared vesicle or micelle can be controlled by extrusion through polycarbonate membranes with a certain pore diameters.

In a preferred embodiment, the vesicle or micelle comprise a detectable marker selected from the group consisting of: fluorophores such as 5-Carboxytetramethylrhodamine, 5-FAM (5-Carboxyfluorescein), 7-Amino-4-methylcoumarin, Bodipy, Fluorescein, Rhodamine 6G, SYTO, semiconductor quantum dots such as CdS, CdSe, CdTe, HgTe, InP, InAs (1-100 nm), metal nanoparticles such as Au, Ag (1-100 nm) and magnetic nanoparticles such as Fe3O4, FeO, Fe2O3 (5-200 nm).

In a preferred embodiment, the detectable marker resides within the vesicle or micelle.

Detectable markers are introduced to improve the sensitivity of detecting oligonucleotide-lipid membrane architectures. The markers may be introduced by extrusion of vesicles in buffer containing the respective marker. Non-introduced markers may be removed by purification steps.

### Target Nucleic Acid

The target nucleic acid may be a synthetic nucleic acid, such as a RNA or DNA oligonucleotide.

In a preferred embodiment, the target nucleic acid is derived from biological material and selected from the group consisting of, an mRNA, microRNAs, rRNA, tRNA and chromosomal DNA and a PCR product. The phrase “derived from biological material” intended to mean that the target nucleic acid is purified or separated from biological material. Nucleic acids of biological material may also be manipulated by e.g. PCR amplification or other means such as to provide a nucleic acid that is still derived from biological material.

### Detection and Quantification

In a preferred embodiment of the method, the method is used for detecting the presence of a particular nucleic acid, e.g. a nucleotide sequence of a certain bacteria or virus. Thus, the method may be used for detecting a particular bacteria or virus or other micro organisms in a sample obtained from a mammal.

The method may also be used for detecting a single nucleotide polymorphism (SNP) in a mammal, such as a human. As mentioned above and illustrated in the examples section, the present method provides very specific base pairing and is consequently well suited for detection of SNPs.

Oligonucleotide-lipid membrane aggregation may be detected by visual inspection of the sample comprising the amphiphilic oligonucleotide, the lipid membrane and the target nucleic acid.

The method may also be used for quantifying the presence of a particular nucleic acid. In this embodiment, the degree of oligonucleotide-lipid membrane aggregation may be monitored by measuring optical density of the sample comprising the amphiphilic oligonucleotide, the lipid membrane and the target nucleic acid. The measured optical density may be compared to a standard curve prepared by measurements on oligonucleotide-lipid membrane aggregation using predetermined amounts of a target nucleic acid.

Detection or quantification may also involve light-scattering techniques or UV-spectroscopy.

### Separation and Purification

The method of constructing oligonucleotide-lipid membrane architectures may also be used for separating the target nucleic acid from other molecules such as other nucleic acids. This may be done, because hybridisation of the target nucleic acid to the amphiphilic oligonucleotide in the presence of a lipid membrane can lead to lipid membrane aggregation. The resulting lipid membrane aggregate can be separated from other molecules in solution e.g. by way of filtration or centrifugation. After separation, the target nucleic acid may be released from the lipid membranes e.g. denaturing agents (high ph), heating, enzymatic cleavage or photocleavage. The target nucleic acid may also be released as a complex with the amphiphilic oligonucleotide by releasing the membrane anchors from the amphiphilic oligonucleotide, e.g. by enzymatic cleavage or photocleavage.

### Immobilisation

In a preferred embodiment of the method of constructing oligonucleotide-lipid membrane architectures, the target nucleic acid is an oligonucleotide that has been or can be immobilised on a solid support.

In another embodiment, the amphiphilic oligonucleotide has been or can be immobilised on a solid support.

Immobilisation may be done by way of a capture oligonucleotide immobilized on the solid support. Immobilisation may also be done by way of a capture group introduced into the target nucleic acid or the amphiphilic oligonucleotide. Exemplary capture groups are amine-groups and carboxylic groups that may be coupled to each other by the use of NHS and EDC. Other capture groups are e.g. antigens, antibodies, biotin etc.

In a preferred embodiment, the solid support is a microarray. When the solid support is a microarray, it may be desirable at least one of the membrane anchors inserts into the lipid membrane irreversibly under the conditions employed. Alternatively, two membrane anchors may be juxtaposed to give a bivalent membrane anchor that inserts irreversibly into a lipid membrane. In such an embodiment, different capture groups may be used for different areas of the microarray allowing directed immobilisation (micropatterning) on the microarray. Preferably, the capture groups in this embodiment are oligonucleotides.

### Other Aspects of the Invention

A second aspect of the invention is the amphiphilic oligonucleotide described above, in various embodiments, in relation to the method of constructing oligonucleotide-lipid membrane architectures. Among other, the amphiphilic oligonucleotide has the interesting characteristic that it can be used for construction of oligonucleotide-lipid membrane architectures and as outlined above it also has characteristics which make interesting for therapeutic use.

A third aspect of the present invention is the lipid membrane described above in various embodiments comprising the amphiphilic oligonucleotide of the invention.

A fourth aspect of the invention is use of the amphiphilic oligonucleotide of the invention for insertion into a lipid membrane.

A fifth aspect of the invention is the amphiphilic oligonucleotide of the invention for use as medicine.

A sixth aspect of the invention is a pharmaceutical composition comprising the amphiphilic oligonucleotide of the invention. In addition to its favourable application in the method of constructing oligonucleotide-lipid membrane architectures, the amphiphilic oligonucleotide of the invention has improved properties in terms of reduced self-aggregation and consequently improved pharmaceutical properties. As explained in the background section, one strategy to improve delivery of oligonucleotides into cells is by conjugation of a lipid moiety to the oligonucleotide. However, this imposes a tendency for aggregation on the oligonucleotide. By introducing two lipid moieties, experiments have shown that this tendency is decreased or removed.

A seventh aspect of the invention is a method of treatment comprising administrating the amphiphilic oligonucleotide of the invention or the pharmaceutical composition comprising the amphiphilic oligonucleotide of the invention to a person in need thereof.

An eight aspect of the invention is a kit for detecting the presence of a target nucleic acid, the kit comprising

### EXAMPLES

### Example 1

### Materials & Methods

### Synthesis of Building Blocks

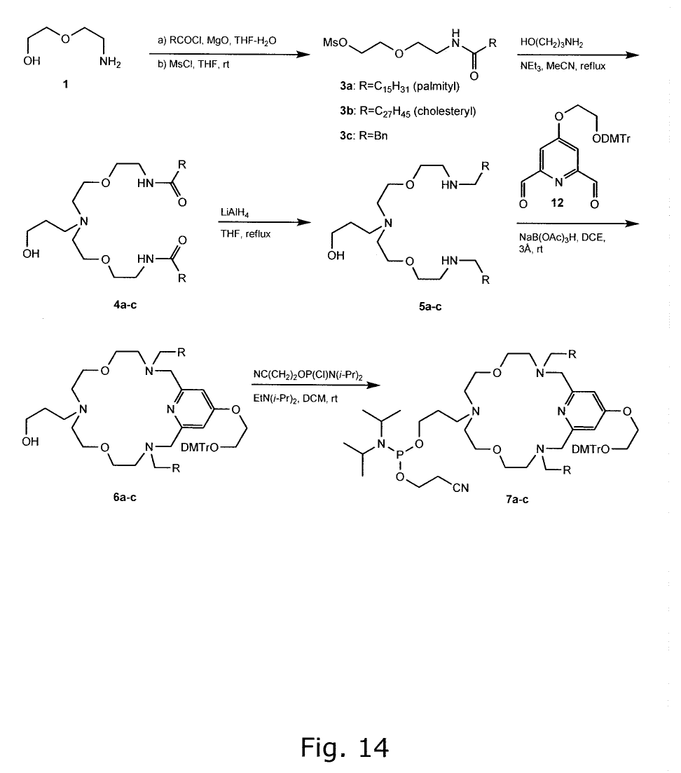

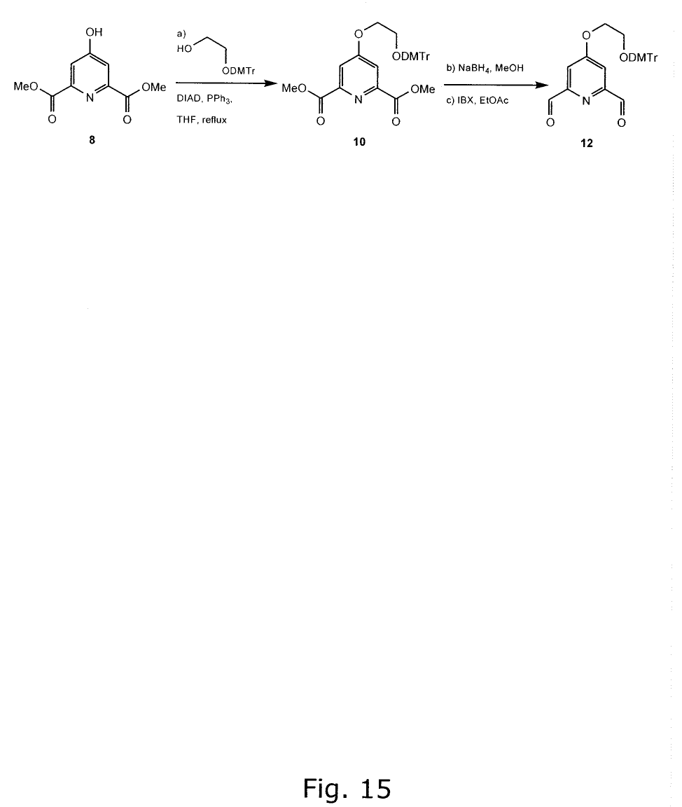

Se FIGS. 14 and 15 for outline.

Synthesis of N-[2-(2-hydroxyethoxy)ethyl]hexadecanamide (2a)—General Procedure: A solution of 2-(2-aminoethoxy)-ethanol (8.21 g, 78 mmol) in water (70 ml) and THF (210 ml) was cooled to 5° C. (ice bath) and MgO (15.71 g, 390 mmol) was added followed by 30 min stirring. Palmitoylchloride (21.43 g, 78 mmol) dissolved in THF (70 ml) was slowly added to the slurry keeping the temperature of the reaction mixture below 10° C. After continuous rapid stirring for 2 h at 5° C. the reaction mixture was filtered and the filtrate evaporated to dryness affording 2a (26.78 g, 100%). 1H-NMR: δ (ppm)=0.85 (t, 3H, CH3), 1.15-1.38 (m, 24H, CH2), 1.62 (m, 2H, CH2), 2.18 (bs, 1H, OH), 2.18 (m, 2H, CH2), 3.48 (m, 2H, CH2), 3.58 (m, 4H, CH2), 3.75 (m, 2H, CH2), 5.86 (bs, 1H, NH). 13C-NMR: δ (ppm)=14.23, 22.80, 25.88, 29.47, 29.50, 29.64 29.78 29.81 32.04 36.89, 39.29, 61.83, 70.14, 72.37, 173.67. MALDI-MS: m/z calcd. for C20H41NO3 [M+Na]+: 366.2979; found: 366.2970.

Synthesis of (3a,8a,9b,10a,13a,14b,20S)—N-[2-(2-hydroxyethoxy)ethyl]cholest-5-ene-3-carboxamide (2b): Dry column FC (CHCl3:THF:HOAc-9:1:0.1) afforded 2b (9.56 g, 74%). 1H-NMR: δ (ppm)=1H-NMR: δ (ppm)=0.67 (bs, 6H, CH3), 0.85-2.52 (m, 44H, CH, CH2, CH3), 3.44-3.47 (m, 2H, NCH2), 3.55-3.60 (m, 4H, OCH2), 3.73-3.76 (m, 2H, HOCH2), 5.29 (m, 2H, CH), 5.98 (m, 1H, NH). 13C-NMR: δ (ppm)=11.98, 18.85, 19.55, 20.99, 22.69, 22.94, 23.97, 24.39, 25.93, 28.13, 28.36, 31.91, 32.03, 35.76, 35.91, 36.32, 37.09, 39.00, 39.20, 39.64, 39.91, 42.42, 46.98, 50.48, 56.32, 56.94, 61.86, 70.12, 70.35, 120.97, 141.56, 176.09. MALDI-MS: m/z calcd for C32H55NO3 [M+Na]+: 524.4074; found: 524.4067.

Synthesis of N-[2-(2-hydroxyethoxy)ethyl]benzamidei (2c): Yield: (56.9 g, 95%). 2c was used without further purification. 1H-NMR: δ (ppm)=3.06 (bs, 1H, OH), 3.58 (m, 2H, CH2), 3.64 (d, 4H, CH2), 3.73 (m, 2H, CH2), 7.05 (bs, 1H, NH), 7.42 (m, 3H, CH), 7.78 (m, 2H, CH). 13C-NMR: δ (ppm)=39.9, 61.7, 70.0, 72.4, 127.2, 128.6, 131.5, 134.5, 168.0. EA: Calculated: C, 63.14%, H, 7.23%, N: 6.69%, O: 22.94% Found: C, 62.66%, H, 7.25%, N, 6.68%. MALDI-MS: m/z calcd for C11H15NO3 [M+Na]+: 232.0944; found: 232.0943.

Synthesis of 2-[2-(palmitoylamino)ethoxy]ethyl methanesulfonate (3a)—General Procedure: A solution of 2a (26.78 g, 78 mmol) in THF (200 ml) was cooled to 5° C. (ice bath), NEt3 (15.76 g, 158 mmol) and mesylchloride (12.3 ml, 158 mmol) were added keeping the temperature of the reaction mixture below 10° C. After continuous rapid stirring for 2 h at 5° C. the reaction mixture was evaporated to dryness and redissolved in CHCl3 (300 ml) followed by a wash with brine and saturated aqueous NaHCO3 solution. The organic layer were collected and evaporated to dryness affording 3a (29.6 g, 90%). 1H-NMR: δ (ppm)=0.88 (t, 3H, CH3), 1.15-1.45 (m, 24H, CH2), 1.62 (m, 2H, CH2), 2.18 (m, 2H, CH2), 3.06 (s, 3H, CH3), 3.46 (m, 2H, CH2), 3.58 (m, 2H, CH2), 3.73 (m, 2H, CH2), 4.38 (m, 2H, CH2), 6.06 (bs, 1H, NH). 13C-NMR: δ (ppm)=14.26, 22.81, 25.87, 29.48, 29.51, 29.65, 29.81, 32.04, 36.79, 38.07, 39.09, 68.81, 68.88, 70.26, 173.56. MALDI-MS: m/z calcd for C21H43NO5S [M+Na]+: 444.2754; found: 444.2736.

Synthesis of 2-(2-{[(3a,8a,9b,10a,13a,14b,20S)-cholest-5-en-3-ylcarbonyl]amino}ethoxy)ethyl methanesulfonate (3b): Yield (11 g, 99%). 1H-NMR: δ (ppm)=1H-NMR: δ (ppm)=0.67 (bs, 6H, CH3), 0.85-2.52 (m, 44H, CH, CH2, CH3), 3.05 (s, 3H, CH3SO2O), 3.43-3.48 (m, 2H, NCH2), 3.56-3.59 (m, 2H, OCH2), 3.71-3.74 (m, 2H, OCH2), 4.36-4.39 (m, 2H, CH3SO2OCH2), 5.33 (m, 2H, CH), 6.03 (m, 1H, NH). 13C-NMR: δ (ppm)=11.99, 18.85, 19.55, 21.00, 22.69, 22.95, 23.96, 24.40, 25.93, 28.14, 28.36, 31.93, 32.05, 35.70, 35.92, 36.32, 37.10, 38.06, 39.00, 39.65, 39.92, 42.43, 46.91, 50.45, 56.31, 56.94, 68.84, 70.25, 120.92, 141.63, 175.98. MALDI-MS: m/z calcd for C33H57NO5S [M+Na]+: 602.3850; found: 602.3852.

Synthesis of 2-(2-benzamidoethoxy)ethyl methanesulfonate (3c): FC(CH2Cl2→CH2Cl2:MeOH 50:1) afforded 3c (40.2 g, 61%). 1H-NMR: δ (ppm)=3.00 (s, 3H, SCH3), 3.68 (s, 4H, CH2), 3.76 (m, 2H, CH2), 4.38 (m, 2H, CH2), 6.79 (bs, 1H, NH), 7.27-7.50 (m, 3H, CH), 7.82 (m, 2H, CH). 13C-NMR: δ (ppm)=37.9, 39.7, 68.8, 70.0, 127.2, 128.6, 131.6, 134.4, 167.7. EA: Calculated: C, 50.16%, H: 5.96%, N, 4.87%, O: 27.84%, S: 11.16%. Found: C, 51.14%, H, 5.95%, N: 5.07%. MALDI-MS: m/z calcd for C12H17NO5S [M+Na]+: 310.0720; found: 310.0716.

Synthesis of 3-{bis-[2-(2-palmitoylamino-ethoxy)-ethyl]-amino}-propan-1-ol (4a)—General Procedure: A mixture of n-propanolamine (1 g, 13.3 mmol), mesylate 3a (16.84 g, 39.93 mmol), triethylamine (7.41 ml, 53.2 mmol) and molecular sieve (3 Å, 2 g) in acetonitrile (60 ml, anhydrous) was refluxed for 36 h. After filtration and removal of the solvent under reduced pressure the residue was dissolved in chloroform, and washed with saturated aqueous NaHCO3 (250 ml). After extraction with dichloromethane the combined organic layers were dried with MgSO4, filtered, and the solvent was removed under reduced pressure. Dry column FCii (CH2Cl2:MeOH=40:1) afforded 4a (5.87 g, 61%). 1H-NMR: δ (ppm)=0.88 (dd, 6H, CH3), 1.15-1.38 (m, 48H, CH2), 1.60-1.74 (m, 6H, CH2), 2.12-2.18 (m, 4H, NCH2), 2.66-2.78 (m, 6H, NCH2), 3.40-3.56 (m, 6H, CH2NHCO, OCH2), 3.83 (m, 2H, HOCH2), 7.23 (bs, 2H, NH). 13C-NMR: δ (ppm)=14.22, 22.79, 25.97, 27.96, 29.25, 29.47, 29.61, 29.68, 29.81, 32.03, 36.60, 39.24, 54.70, 55.83, 64.89, 68.25, 70.31, 173.59. MALDI-MS: m/z calcd for C43H87N3O5 [M+Na]+: 748.6538; found: 748.6508.

Synthesis of (3a,17a)-N-[13-[(3a,8a,9b,10a,13a,14b,20S)-cholest-5-en-3-yl]-6-(3-hydroxypropyl)-13-oxo-3,9-dioxa-6,12-diazatridec-1-yl]cholest-5-ene-3-carboxamide (4b): Dry column FC(CHCl3→CHCl3:MeOH-80:1) afforded 4b (3.61 g, 45%). 1H-NMR: δ (ppm)=0.67 (bs, 6H, CH3), 0.85-2.12 (m, 90H, CH, CH2, CH3), 2.42-2.78 (m, 6H, NCH2), 3.40-3.55 (m, 12H, NCH2, OCH2), 3.80 (m, 2H, HOCH2), 5.30 (m, 2H, CH), 7.16 (m, 2H, NH). MALDI-MS: m/z calcd for C67H115N3O5 [M+Na]+: 1041.88; found: 1041.88.

Synthesis of 3-{bis-[2-(2-benzoylamino-ethoxy)-ethyl]-amino}-propan-1-ol (4c): FC(CH2Cl2:MeOH:NH3 (aq. 25%)=20:1:0→5:1:0.1) afforded 4c (3.65 g, 40 30%). 1H-NMR: δ (ppm)=1.63 (m, 2H, CH2), 2.67 (t, 4H, CH2), 2.72 (t, 2H, CH2), 3.58 (m, 14H, CH2), 7.38 (m, 6H, CH), 7.73 (bs, 2H, 2×NH), 7.81 (m, 4H, CH). 13C-NMR: δ (ppm)=27.9, 40.0, 54.1, 55.8, 64.5, 68.2, 70.2, 127.3, 128.3, 131.2, 134.8, 167.9. MALDI-MS: m/z calcd for C25H35N3O5 [M+Na]+: 480.24689; found: 480.24720.

Synthesis of 3-(bis{2-[2-(hexadecylamino)ethoxy]ethyl}amino)propan-1-ol (5a)—General Procedure: A solution of 4a (5.87 g, 8.08 mmol) in THF (50 ml) was added slowly at 0° C. to a solution of LiAlH4 (0.31 g, 1M solution in THF, 24.25 mmol). The reaction mixture was stirred for 1 h at 0° C., and then refluxed for 16 h. After cooling to 0° C. and dilution to double volume with THF a 5% aquous solution of NaOH (20 ml) was added dropwise and the solution was stirred for additional 30 min at 0° C. and 90 min at rt. Filtration, rinsing with hot THF (300 ml), removal of the solvent under reduced pressure, coevaporation with toluene and dry column FCii (CH2Cl2:MeOH:NH3 (aq. 25%)-10:1:0.1) afforded 5a (4.8 g, 85%). 1H-NMR: δ (ppm)=0.88 (t, 6H, CH3), 1.15-1.48 (m, 54H, CH2), 1.48 (m, 4H, CH2), 1.66 (m, 2H, CH2), 2.59 (m, 4H, CH2), 2.68-2.79 (m, 10H, CH2), 3.49-3.58 (m, 8H, CH2), 3.75 (m, 4H, CH2). 13C-NMR: δ (ppm)=14.23, 22.80, 27.55, 28.64, 29.48, 29.81, 30.26, 32.06, 49.54, 50.24, 54.09, 55.40, 63.88, 69.20, 70.71. MALDI-MS: m/z calcd for C43H91N3O3 [M+Na]+: 720.6953; found: 720.6980.

Synthesis of 3-{[2-(2-{[(3a,17a)-cholest-5-en-3-ylmethyl]amino}ethoxy)ethyl][2-(2-{[(3a,8a,9b,10a,13a,14b,205)-cholest-5-en-3-ylmethyl]amino}ethoxy)ethyl]amino}propan-1-ol (5b): Yield: (2.30 g, 99%). 1H-NMR: δ (ppm)=0.67 (bs, 6H, CH3), 0.85-2.12 (m, 90H, CH, CH2, CH3), 2.48 (m, 4H, NCH2), 2.70-2.76 (m, 10H, NCH2), 3.53-3.56 (m, 8H, OCH2), 3.73-3.77 (m, 2H, CH2), 5.29 (m, 2H, CH). 13C-NMR: δ (ppm)=12.01, 18.87, 19.63, 21.07, 22.71, 22.96, 23.99, 24.43, 27.36, 28.15, 28.39, 28.61, 32.04, 35.95, 36.35, 37.51, 37.83, 39.40, 39.66, 39.99, 42.45, 49.66, 50.56, 54.08, 55.43, 56.32, 56.70, 56.97, 63.94, 69.22, 70.57, 119.78, 142.84. MALDI-MS: m/z calcd for C67H119N3O3 [M+Na]+: 1036.9144; found: 1036.9109.

Synthesis of 3-{bis-[2-(2-benzylamino-ethoxy)-ethyl]-amino}-propan-1-ol (5c): Yield (3.40 g, 99%). 5c was used without further purification. Analytical column (CH2Cl2:MeOH:NH3 (aq. 25%)-20:1:0→10:1:0→10:1:0.1). 1H-NMR: δ (ppm)=1.64 (m, 2H, CH2), 2.71 (m, 6H, CH2), 2.78 (t, 4H, CH2), 3.54 (m, 8H, CH2), 3.70 (t, 2H, CH2), 3.80 (s, 4H, CH2), 7.23-7.34 (m, 10H, CH). 13C-NMR: δ (ppm)=28.4, 48.8, 53.9, 54.1, 55.4, 70.0, 69.1, 70.4, 127.1, 128.4, 128.5, 140.0. MALDI-MS: m/z: calcd for C25H39N3O3 [M+Na]+: 452.2884; found: 452.2883.

Synthesis of 3-[19-{2-[bis(4-methoxyphenyl)(phenyl)methoxy]ethoxy}-3,15-dihexadecyl-6,12-dioxa-3,9,15,21-tetraazabicyclo[15.3.1]henicosa-1(21),17,19-trien-9-yl]propan-1-ol (6a)—General Procedure: Sodium triacetoxy borohydride (1.21 g, 5.72 mmol) was added to a solution of 12 (0.71 g, 1.43 mmol) and molecular sieves (3 Å, 2 g) in 1,2-dichloroethane (30 ml, anhydrous). After addition of compound 5a (1.00 g, 1.43 mmol) in 1,2-dichloroethane (30 ml, anhydrous) the reaction mixture was stirred for 48 h at rt. Then saturated sodium bicarbonate solution (30 ml) was added, and the mixture stirred for additional 2 h. After extraction with chloroform the combined organic layers were dried with MgSO4, filtered, and the solvent was removed under reduced pressure. FC (CHCl3:MeOH:NEt3→10:1:0.1) afforded 6a (0.68 g, 41%). 1H-NMR: δ (ppm)=0.87 (m, 6H, CH3), 1.06-1.42 (m, 52H, CH2), 1.53 (m, 6H, CH2), 2.51-2.71 (m, 14H, NCH2), 3.31-3.44 (m, 8H, OCH2), 3.70-3.78 (m, 14H, CH2, OCH3), 4.18 (dd, 2H, CH2), 6.81-7.49 (m, 15H, CH). 13C-NMR: δ (ppm)=14.27, 22.83, 27.67, 28.55, 29.50, 29.84, 32.06, 53.16, 54.32, 55.33, 56.35, 56.91, 61.20, 62.31, 64.12, 67.32, 69.10, 69.60, 86.29, 108.06, 113.23, 126.89, 127.94, 128.33, 130.21, 136.18, 144.93, 158.60, 161.37, 166.07. MALDI-MS: m/z calcd for C73H118N407 [M+Na]+: 1185.8893; found: 1185.8873.

Synthesis of 3-[19-{2-[bis(4-methoxyphenyl)(phenyl)methoxy]ethoxy}-3-[(3a,8a,9b,10a,13a,14b, 20S)-cholest-5-en-3-ylmethyl]-15-[(3b,17a)-cholest-5-en-3-ylmethyl]-6,12-dioxa-3,9,15,21-tetra-aza-bicyc-lo[15.3.1]henicosa-1(21),17,19-trien-9-yl]propan-1-ol (6b): FC(CHCl3:MeOH:NEt3→20:1:0.1) afforded 6b (1.49 g, 46%). 1H-NMR: δ (ppm)=0.67 (bs, 6H, CH3), 0.85-2.22 (m, 90H, CH, CH2, CH3), 2.42-2.67 (m, 14H, NCH2), 3.32-3.44 (m, 8H, OCH2), 3.70-3.78 (m, 14H, CH2, NCH2, OCH3), 4.18 (m, 2H, CH2), 5.30 (m, 2H, CH), 6.81-7.49 (m, 15H, CH). 13C-NMR: δ (ppm)=11.99, 18.85, 19.66, 21.06, 22.70, 22.95, 23.94, 24.41, 24.53, 27.72, 28.14, 28.37, 28.53, 32.02, 35.92, 36.32, 37.64, 38.25, 38.39, 39.55, 39.65, 39.96, 42.20, 50.61, 53.54, 54.38, 55.30, 56.27, 56.43, 56.95, 61.76, 62.33, 63.59, 64.14, 67.24, 69.07, 69.45, 86.28, 108.02, 113.21, 119.76, 126.88, 127.94, 128.34, 130.19, 136.19, 143.12, 144.89, 158.59, 161.45, 165.99. MALDI-MS: m/z calcd for C97H146N4O7 [M+Na]+: 1502.1084; found: 1502.1050.

Synthesis of 3-[3,15-dibenzyl-19-{2-[bis(4-methoxyphenyl)(phenyl)methoxy]ethoxy}-6,12-dioxa-3,9,15,21-tetraazabicyclo[15.3.1]henicosa-1(21),17,19-trien-9-yl]propan-1-ol (6c): FC (CHCl3:MeOH:NEt3→10:1:0.1) afforded 10 (1.36 g, 65%). 1H-NMR: δ (ppm)=1.60 (m, 2H, CH2), 2.56 (t, 4H, CH2), 2.62 (t, 2H, CH2), 2.77 (t, 4H, CH2), 3.36 (t, 4H), 3.46 (m, 6H, CH2), 3.68-3.80 (m, 16H, CH2, OCH3), 4.16 (t, 2H, CH2), 6.81-6.84 (m, 6H, CH), 7.20-7.49 (m, 19H, CH), 13C-NMR: δ (ppm)=28.5, 52.9, 54.4, 55.3, 56.3, 60.5, 60.6, 62.3, 64.1, 67.2, 69.2, 69.5, 86.3, 108.0, 113.2, 126.9, 127.1, 127.9, 128.3, 128.4, 129.0, 130.2, 136.2, 139.6, 144.9, 158.6, 160.9, 166.0. MALDI-MS: m/z calcd for C55H66N4O7 [M+Na]+: 917.4824; found: 917.4817.

Synthesis of 3-[19-{2-[bis(4-methoxyphenyl)(phenyl)methoxy]ethoxy}-3,15-dihexadecyl-6,12-dioxa-3,9,15,21-tetraazabicyclo[15.3.1]henicosa-1(21),17,19-trien-9-yl]propyl-2-cyanoethyldiisopropyl-amidophosphite (7a)—General Procedure: 2-cyanoethyl diisopropyl-amidochloridophosphite (54.4 mg, 0.206 mmol) is added slowly at 0° C. to a solution of 6a (200 mg, 0.171 mmol), and diisopropylethylamine (53.3 mg, 0.412 mmol) in dichloromethane (5 ml, anhydrous). After stirring for 1 h at 0° C. the reaction mixture was washed with saturated sodium bicarbonate solution and extracted with dichloroethane. The combined organic layers were dried with MgSO4, filtered, and solvents removed under reduced pressure. FC (EE:NEt3-10:0.2) afforded 7a (141.5 mg, 60%). 1H-NMR: δ (ppm)=0.87 (m, 6H, CH3), 1.05-1.42 (m, 68H, CH2), 1.48-1.69 (m, 6H, CH2), 2.50-2.70 (m, 16H, CH2CN, NCH2), 3.25-3.81 (m, 26H, OCH2, OCH3, NCH2, CH2), 4.18 (dd, 2H, OCH2), 6.81-7.55 (m, 15H, CH). 13C-NMR: δ (ppm)=14.26, 20.42, 20.51, 22.82, 24.68, 24.71, 24.77, 24.80, 27.67, 29.00, 29.10, 29.50, 29.83, 32.06, 43.03, 43.19, 53.01, 53.35, 54.46, 55.32, 57.01, 58.24, 58.50, 61.24, 61.92, 62.15, 62.29, 67.33, 69.58, 69.80, 86.29, 108.09, 113.23, 126.89, 127.94, 128.33, 130.20, 136.19, 144.93, 158.61, 161.46, 166.00. 31P-NMR: δ (ppm)=148.32. HR-ESI-MS: m/z calcd for C82H135N6O8P [M+2H]2+: 682.5112; found: 682.5120.

Synthesis of 3-[19-{2-[bis(4-methoxyphenyl)(phenyl)methoxy]ethoxy}-3-[(3a,8a,9b,10a,13a,14b,205)-cholest-5-en-3-ylmethyl]-15-[(3b,17a)-cholest-5-en-3-ylmethyl]-6,12-dioxa-3,9,15,21-tetraazabicyclo-[15.3.1]henicosa-1(21),17,19-trien-9-yl]propyl-2-cyanoethyldiiso-propylamidophosphite (7b): FC (EE:NEt3-10:0.2) afforded 7b (255 mg, 56%). 1H-NMR: δ (ppm)=0.67 (bs, 6H, CH3), 0.85-2.22 (m, 96H, CH, CH2, CH3), 2.42-2.67 (m, 16H, NCH2), 3.24-3.88 (m, 26H, CH2, NCH, NCH2, OCH2, OCH3), 4.18 (m, 2H, CH2), 5.30 (m, 2H, CH), 6.81-7.49 (m, 15H, CH). 13C-NMR: δ (ppm)=12.01, 18.86, 19.57, 19.67, 20.43, 20.52, 21.08, 21.46, 22.70, 22.96, 23.95, 24.42, 24.71, 24.78, 27.80, 28.15, 28.38, 29.05, 29.15, 32.04, 35.93, 36.33, 37.65, 38.28, 38.42, 39.57, 39.66, 39.98, 42.43, 43.03, 43.29, 50.63, 53.03, 53.79, 54.53, 56.28, 56.96, 58.26, 58.51, 61.88, 62.16, 62.34, 63.70, 67.25, 69.51, 69.79, 86.29, 108.11, 113.22, 119.77, 126.89, 127.95, 128.34, 130.19, 136.20, 143.13, 144.90, 158.59, 161.56, 165.97. 31P-NMR: δ (ppm)=148.30, 149.38 (<5%).

Synthesis of 2-cyanoethyl-3-[3,15-dibenzyl-19-{2-[bis(4-methoxyphenyl)-(phenyl)methoxy]-ethoxy}-6,12-dioxa-3,9,15,21-tetraazabicyclo[15.3.1]henicosa-1(21),17,19-trien-9-yl]propyldiiso-propyl-amidophosphite (7c): FC (EE:NEt3-10:0.2) afforded 7c (305 mg, 83%). 1H-NMR: δ (ppm)=1.16 (t, 12H, CH3), 1.68 (m, 2H, CH2), 2.56 (m, 8H, CH2), 2.76 (t, 4H, CH2), 3.34 (t, 4H, CH2), 3.44 (m, 6H, CH2), 3.53-3.81 (m, 20H, CH2), 4.15 (t, 2H, CH2), 6.82 (m, 6H, CH), 7.21-7.40 (m, 19H, CH). 13C-NMR: δ (ppm)=20.49, 24.72, 24.80, 29.10, 43.05, 43.21, 52.94, 53.05, 54.56, 55.35, 58.38, 60.58, 60.72, 62.06, 62.28, 67.27, 69.46, 69.83, 86.33, 108.06, 113.25, 126.93, 127.17, 127.96, 128.34, 128.43, 129.05, 130.21, 136.17, 139.59, 144.91, 158.63, 161.00, 165.96. 31P-NMR: δ (ppm)=148.3 (s).

Synthesis of 4-[2-(4,4′-dimethoxytrityl)-oxy-ethoxy]-pyridine-2,6-dicarboxylic acid dimethyl ester (10): Ethyleneglycol (39.7 g, 640 mmol, 36 ml) and N,N-dimethoxy-4-amino-pyridine (977 mg, 8 mmol) were dissolved in dichloromethane (200 ml, anhydrous) and cooled to 0° C. Under Ar-atmosphere diisopropylethylamine (24.8 g, 192 mmol, 33.5 ml) and then 4,4′-dimethoxytritylchloride (54.2 g, 160 mmol) in dichloromethane (500 ml, anhydrous) were added slowly. After 3 h stirring at rt the solvent was removed under reduced pressure. FC(CHCl3: NEt3-20:0.1) afforded 2-(4,4′-dimethoxytrityl)-ethyleneglycol (42.2 g, 72%) 9. 1H-NMR: δ (ppm)=3.25 (t, 3JH, H=4.7 Hz, 2H, CH2), 3.73 (t, 3JH,H=4.7 Hz, 2H, CH2), 3.78 (s, 6H, CH3), 6.82 (m, 4H, CH), 7.23-7.34 (m, 7H, CH), 7.44 (d, 2H, CH), 13C-NMR: δ (ppm)=55.3, 62.5, 64.7, 86.2, 113.3, 126.9, 128.0, 128.3, 130.2, 136.2, 145.0, 158.6. MALDI-MS m/z: calcd for C23H24O4 [M+Na]+: 387.1567; found: 387.1576. Diisopropyl azodicarboxylate (21.25 g, 105.12 mmol, 20.28 ml) was added under Ar-atmosphere at 0° C. and in the dark to a solution of triphenylphosphine (27.57 g, 105.12 mmol) in THF (150 ml, anhydrous). After stirring at 0° C. for 30 min, first 2-(4,4′-dimethoxytrityl)-ethyleneglycol (31.92 g, 87.59 mmol) in THF (100 ml, anhydrous) and then 4-(hydroxy-ethoxy)-pyridine-2,6-dicarboxylic acid dimethyl ester hydrochlorid 8 (21.50 g, 87.05 mmol) in THF (200 ml, anhydrous) were added at 0° C. After reflux for 20 h the solvent was removed under reduced pressure. Dry column (toluene:CHCl3: NEt3-5:4:0.1) afforded 10 (37.29 g, 77%). 1H-NMR: δ (ppm)=3.50 (t, 2H, CH2), 3.79 (s, 6H, CH3), 4.02 (s, 6H, CH3), 4.29 (t, 2H, CH2), 6.83 (m, 4H, CH), 7.20-7.35 (m, 7H), 7.45 (d, 2H), 7.86 (s, 2H, CH). 13C-NMR: δ (ppm)=53.4, 55.3, 62.0, 68.6, 86.6, 113.3, 114.8, 127.0, 128.0, 128.2, 130.2, 135.9, 144.7, 149.9, 158.7, 165.3, 167.2. MALDI-MS m/z: calcd for C32H31NO8 [M+Na]+: 580.1942; found: 580.1937.

Synthesis of 4-[2-(4,4′-dimethoxytrityl)-oxy-ethoxy]-2,6-bis-hydroxymethyl-pyridine (11): NaBH4 (21 g, 552 mmol) was added at 0° C. in small portions to a solution of the crude dimethyl ester 10 (38.1 g, max 43 mmol) in methanol (300 ml, anhydrous). After stirring for 6 h at rt, first the reaction mixture was carefully diluted with water (50 ml), then NaHCO3 (sat. aqu. solution, 50 ml) was added slowly. Some of the methanol (˜200 ml) was removed under reduced pressure. The residue was extracted with chloroform, and the combined organic layers were washed with saturated sodium bicarbonate solution and water. After drying with MgSO4, filtration and removal of the solvent under reduced pressure, the crude was purified with FC. FC(CHCl3:MeOH:Et3N=50:1:0.1→10:1:0.1) afforded 11 (16.87 g, 78% over 2 steps). 1H-NMR: δ (ppm)=3.44 (t, 2H, CH2), 3.78 (s, 6H, CH3), 4.17 (t, 2H, CH2), 4.69 (s, 4H, CH2), 6.74 (s, 2H, CH), 6.81 (m, 4H, CH), 7.20-7.35 (m, 7H, CH), 7.44 (d, 2H, CH). 13C-NMR: δ (ppm)=55.4, 62.1, 64.6, 67.7, 86.4, 105.9, 113.3, 127.0, 128.0, 128.3, 130.2, 136.0, 144.8, 158.6, 160.5, 166.6. MALDI-MS: m/z calcd for C30H31NO6 [M+Na]+: 524.2044; found: 524.2021.

Synthesis of 4-[2-(4,4′-dimethoxytrityl)-oxy-ethoxy]-pyridine-2,6-dicarbaldehyde (12): To a solution of dialcohol 11 (234 mg, 0.467 mmol) and pyridine (122 mg, 1.54 mmol) in ethylacetate (anhydrous, 5 ml) was added IBX (432 mg, 1.54 mmol) at 80° C. After stirring for 90 min at 80° C. the reaction mixture was cooled down to 0° C., filtered, washed with ethylacetate, and the solvent was removed under reduced pressure. FC(CHCl3:MeOH:NEt3-20:1:0.1) afforded 12 (200 mg, 86%). 1H-NMR: δ (ppm)=3.51 (t, 2H, CH2), 3.78 (s, 6H, CH3), 4.30 (t, 2H, CH2), 6.82 (d, 4H, CH), 7.25-7.33 (m, 7H, CH), 7.43 (d, 2H, CH), 7.68 (s, 2H, CH), 10.11 (s, 2H, CH). 13C-NMR: δ (ppm)=55.4, 61.9, 68.8, 86.6, 111.8, 113.3, 127.0, 128.0, 128.2, 130.2, 135.8, 144.7, 154.9, 158.6, 167.2, 192.5. MALDI-MS: m/z calcd for C30H27NO6 [M+Na]+: 520.1731; found: 520.1721.

### Materials:

1-palmitoyl-2-oleoyl-sn-glycero-3-phosphocholine (POPC, lyophilized powder) was purchased from Avanti Polar Lipids and 1,1′-dioctadecyl-3,3,3′,3′-tetramethylindocarbocyanine perchlorate (DiI-C18) and 3,3′-dioctadecyloxacarbocyanine perchlorate (DiO—C18) from Invitrogen/Molecular Probes. 2,2′-(1,8-Dihydroxy-3,6-disulfonaphthylene-2,7-bisazo)bisbenzene arsonic acid (arsenazo III) and all other chemicals were obtained from Sigma in the purest form available. Ultrapure Milli-Q water was used in all experiments. 4-(2-Hydroxyethyl)piperazine-1-ethanesulfonic acid (HEPES) buffer (10 mM HEPES, 110 mM Na⁺) was prepared by mixing appropriate amounts of HEPES, HEPES sodium salt and NaCl followed by correction of pH to 7.0. The chemicals for DNA synthesis were purchased from Glen Research. All solvents (HPLC grade) and reagents were purchased commercially and used without further purification. Muscovite mica (75 mm×25 mm×200 μm sheets) was from Plano GmbH, Germany. Mica sheets of 10 mm×10 mm were preglued onto round (0.17 mm, Ø24 mm) microscope coverslips using a transparent and biocompatible silicone glue (MED-6215, Nusil Technology, Santa Barbara, Calif.). Immediately prior to use, the mica was cleaved with a knife leaving a thin and highly transparent mica film on the coverslip.

### Measurement of Transition Temperatures:

Transition analyses were carried out on a Perkin Elmer UV/VIS spectrometer Lambda 30 with a PTP-6 (Peltier Temperature Programmer) device by using PE TempLab 2.0 and UV Winlab 2.8. Melting temperatures (Tm, in ° C.) were determined as a first derivative of thermal denaturation curves, which were obtained by recording absorbance at 260 nm as a function of temperature at a rate of 0.5° C. min-1 for measurements with liposomes and with 1° C. min-1 for measurements without liposomes. The solutions were heated to 90° C., maintained for 5 min at this temperature, and then gradually cooled before performing thermal denaturation experiments. All melting temperatures are reported with an uncertainty of ±1° C., as determined from multiple experiments.

Experimental procedure for liposome assembly: In a typical experiment we use POPC (1-palmitoyl-2-oleoyl-sn-glycero-3-phosphocholine) liposomes with an average diameter of 65 nm±15 nm and a lipid concentration of 10 mM, prepared by extrusion through 50 nm filters, a final concentration of 0.5 mM is used for all experiments. Addition of lipophilic probe DNA in HEPES buffer solution (pH 7.0, 110 mM Na+, 62 nM probe strand) followed by addition of the target sequence initiates assembly. The total sample volume was 1 ml. Experiments with lower target concentrations (target concentration<100 nM) produce stable aggregates as observed by light scattering, but without macroscopic precipitation, even after prolonged time (24 h).

### Experiments Using Serum and Yeast Cell Extracts:

10 μl of FBS serum pH 6.9-7.8 pH was added to the solution of probe and target strand followed by addition of Hepes buffer to a total volume of 1 ml according to the standard procedure for liposome assembly.

10 μl of yeast cell extract was added to the solution of probe and target strand followed by addition of Hepes buffer to a total volume of 1 ml according to the standard procedure for liposome assembly.

### Preparation of POPC Liposomes:

POPC was suspended in aqueous HEPES buffer at 10 mM lipid concentration and stirred to a uniform milky solution. Liposomes were prepared by repeated extrusion (10 times) through double polycarbonate filters with a 50 nm pore size using compressed N2 (30 bar) and a LIPEX™ Extruder from Northern Lipids. DiO—C18-labeled liposomes were prepared by co-solubilization of POPC and DiO—C18 in CHCl3 followed by evaporation of CHCl3 and hydration in HEPES buffer prior to extrusion.

### Preparation of Supported Membranes:

Supported membranes were prepared by hydration of spincoated lipid films as described previously. The procedure consists of the following steps: To prepare a dry spincoated POPC film on mica a stock solution of 10 mM POPC containing 0.7% DiO—C18 in hexane/methanol (97:3 volume ratio) was used. A droplet (30 μL) of this lipid stock solution was then applied to freshly cleaved mica with a size of 10 mm×10 mm and immediately thereafter spun on a Chemat Technology, KW-4A spin-coater at 3000 rpm for 40 s. The sample was placed under vacuum for 10-15 hours to ensure complete evaporation of solvents. The dry spin coated film was subsequently hydrated by immersing the sample in HEPES buffer followed by heating in an oven at 55° C. for 1 hour. The sample surface was flushed with HEPES buffer at 55° C. which left a single and uniform membrane on the mica surface. After this, the buffer was replaced 10 times to remove membrane fragments in solution.

### Experimental Conditions for DNA Controlled Tethering of Liposomes to a Supported POPC Membrane:

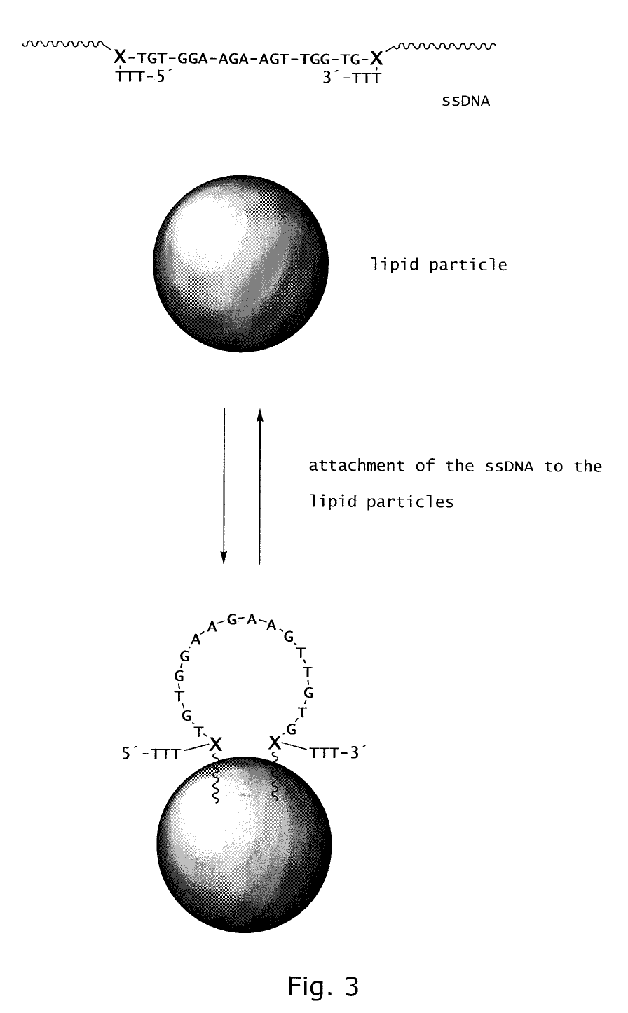

In a typical experiment for DNA controlled tethering of liposomes to a surface, a supported POPC membrane containing fluorescent dye (DiO—C18, 0.5%) was first prepared on a mica surface by spincoating according to our recent protocol. Successful membrane formation was confirmed by independent fluorescence imaging. Subsequently, the membrane was exposed to probe DNA which couples to the membrane by insertion of the alkyl chains. Liposomes labeled with a second fluorophore (DiI-C18, 0.5%) were subsequently added, but as expected no adhering to the supported membrane was observed. Finally, the target strand was added leading to duplex formation and tethering of liposomes to the surface within a time of 1-2 hours (FIG. 3). However, the assembly occurs to a large extend (ca. 90%) within the first 15 min.

### Oligonucleotide Synthesis and MALDI-TOF Analysis of DNA-Conjugates:

Standard procedures for automated DNA synthesis have been used except pyridinium hydrochloride as activator reagent and coupling times of 20 min. Incorporation of monomers into oligodeoxynucleotides (ONs) was performed on a 0.2 μmol scale and was followed by purification of the ONs by HPLC as previously described. MALDI-TOF analysis was performed on a Voyager Elite Biospectrometry Research Station from PerSeptive Biosystems.

### Fluorescence Microscopy:

Epi-fluorescence microscopy of supported membranes coupled to liposomes was performed with the sample placed with the lipid side facing up in a custom made microscope chamber (2 ml volume) and placed on a Nikon TE2000 inverted microscope using an oil immersion objective (Plan Apo, 60×NA=1.4, Nikon). Fluorescence excitation was done using a Xenon lamp with monochromator (Polychrome V, Till Photonics, Gräfelfing, Germany) as well as G-2A (DiI) and B-2A (DiO) filtercubes from Nikon. Images were recorded with a high sensitivity CCD camera (Sensicam em, 1004×1002 pixels, PCO-imaging, Kelheim, Germany) and operated with Camware software (PCO). The experiment was performed in a total volume of 1 ml HEPES buffer (10 mM HEPES, 110 mM Na+, pH 7.0) with a concentration of 100 nM for each strand (DNA 3′-ACACCTTCTTCAACCAC:5′-TTTXTGTGGAAGAAGTTGGTGXTTT). Successful membrane formation was confirmed by independent fluorescence imaging. Thereafter liposomes labeled with a second fluorophor (DiI-C18, 0.5%) and probe strand were added, but without liposomes adhering to the supported membrane. Finally, the target strand was added leading to duplex formation and attachment of liposomes to the surface within a time of 1-2 hours. However an almost complete attachment (immobilization on the supported membrane) was observed already after 15 min. Thermal denaturation of liposome aggregates could thereby be visualized directly by fluorescence. Heating to above the denaturation temperature of the duplex (Tm=46° C.), led to complete release of the attached liposomes into solution and re-aggregation upon cooling below the Tm.

### Preparation of Phospholipid Vesicles.

The phospholipids used were 1,2-dimyristoyl-sn-phosphatidylcholine (DMPC) and 1-palmitoyl-2-oleoyl-sn-phosphatidylcholine (POPC) (Northern Lipids, Vancouver BC); the lipids were obtained in powder form. For each sample, a single variety of phospholipid was hydrated using purified water from a Milli-Q Plus water purification system (Millipore, Bedford Mass.), in ratios of 1-200 mg of phospholipids per milliliter of water. Water from a Milli-Q Plus filtration system was used to keep the concentration of contaminants in the vesicle solution negligible. This ensures that the vesicles will swell to spherical shapes after extrusion. The pre-extruded vesicle suspension was then extruded through two polycarbonate membranes with nominal pore diameters of 50, 100, or 200 nm using an extruder (Lipex Biomembranes, Vancouver, BC) by applying a pressure gradient as provided by prepurified, compressed N2 gas. The area of the membrane available for extrusion was 4.15 cm2. Water from an external water bath was circulated through the extruder in order to control the extrusion temperature. DMPC vesicles were extruded at 40° C. and POPC vesicles were extruded at 25° C. so that both lipids were at temperatures well above their gel-transition temperatures. The vesicle suspension was re-extruded a minimum of 10 times or until the average flow rate became constant on consecutive extrusions. The average flow rate of the extruded suspension was measured.

The size of vesicles can be controlled e.g. by choosing different polycarbonate membranes with nominal pore diameters of 50, 100, or 200 nm using an extruder (Lipex Biomembranes, Vancouver, BC) by applying a pressure gradient as provided by prepurified, compressed N2 gas.

### Example 2

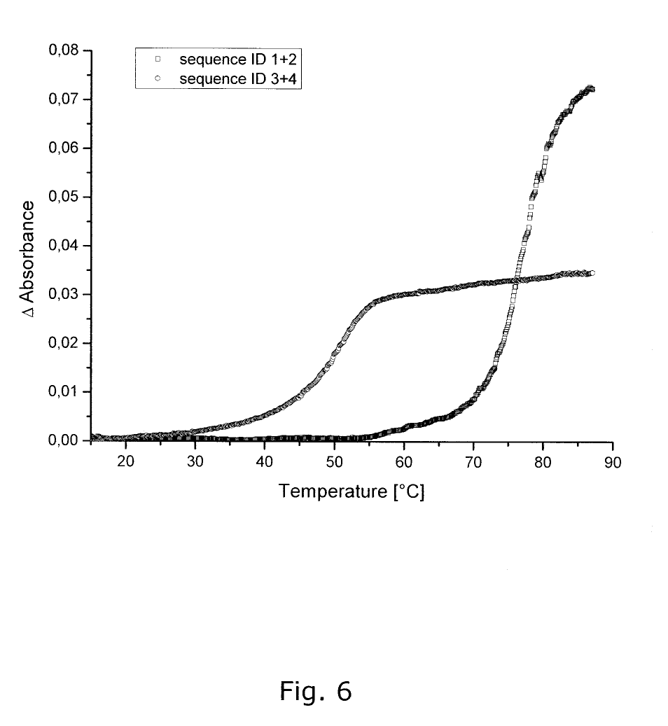

Thermal denaturation curves was recorded in a UV experiment at 260 nm wavelength showing hybridisation of sequence ID1 and ID2 compared to the hybridisation of sequence ID3 and ID4 (FIG. 6)

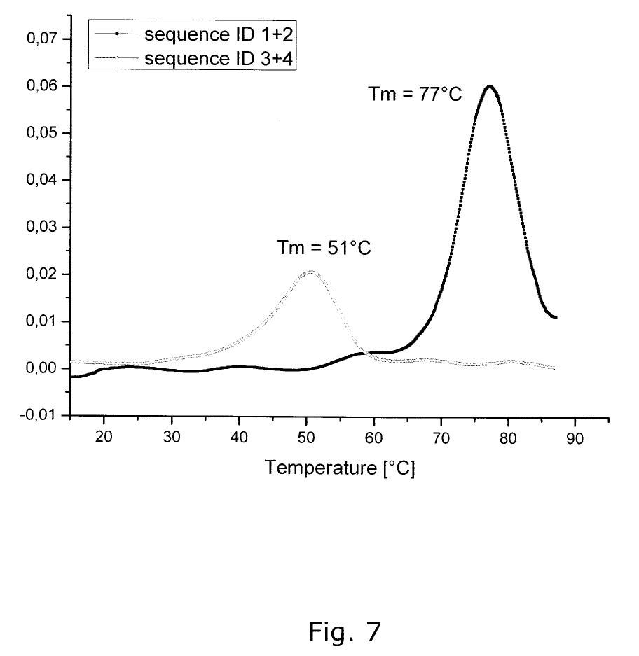

First derivatives of the thermal denaturation curves recorded in a UV experiment at 260 nm wavelength showing hybridisation of sequence ID1 and ID2 compared to the hybridisation of sequence ID3 and ID4 (FIG. 7)

### Example 3

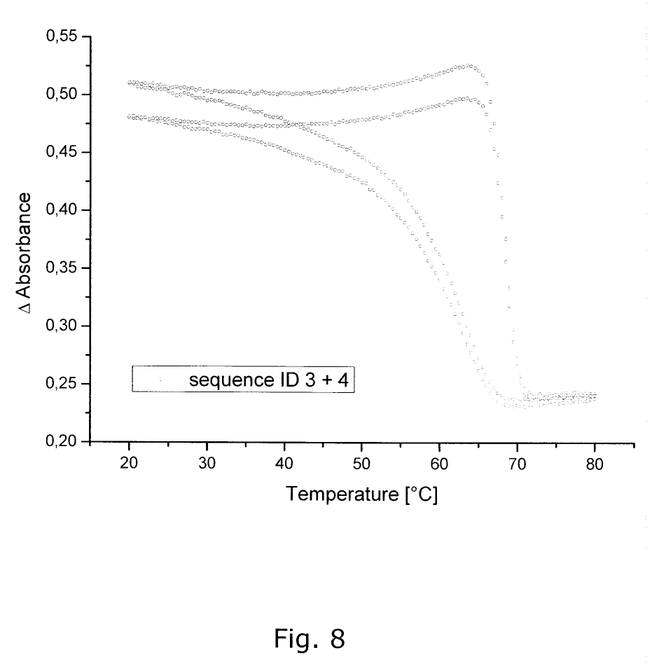

Thermal denaturation curves recorded in a UV experiment at 260 nm wavelength showing hybridisation of sequence ID1 and ID2 compared to the hybridisation of sequence ID3 and ID4 and run in two consecutive cycles from 15° C. to 80° C. (FIG. 8). The results show the reproducibility of the assembly and disassembly process of the bilayers used (vesicles).

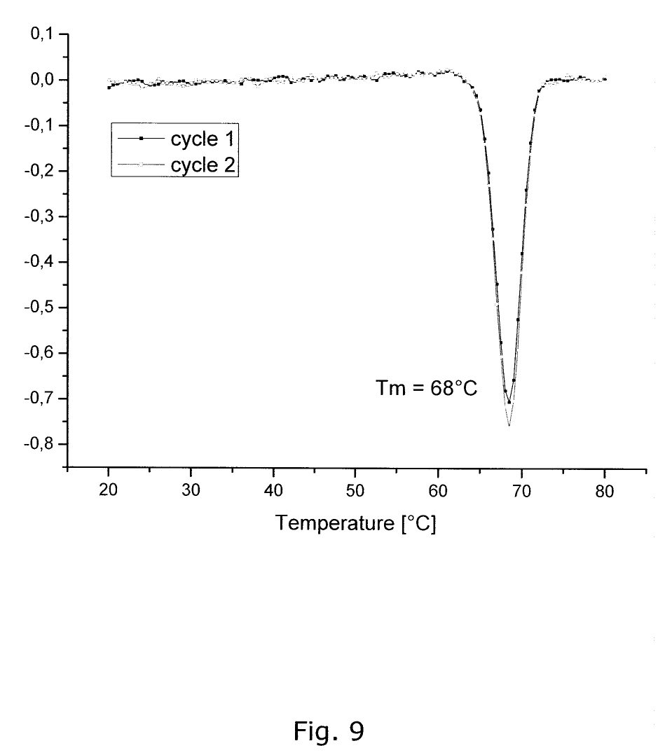

First derivative of the thermal denaturation curves recorded in a UV experiment at 260 nm wavelength showing hybridisation of sequence ID1 and ID2 compared to the hybridisation of sequence ID3 and ID4 and run in two consecutive cycles from 15° C. to 80° C. (FIG. 9). The results again show the reproducibility of the assembly and disassembly process of the bilayers used (vesicles).

### Example 4

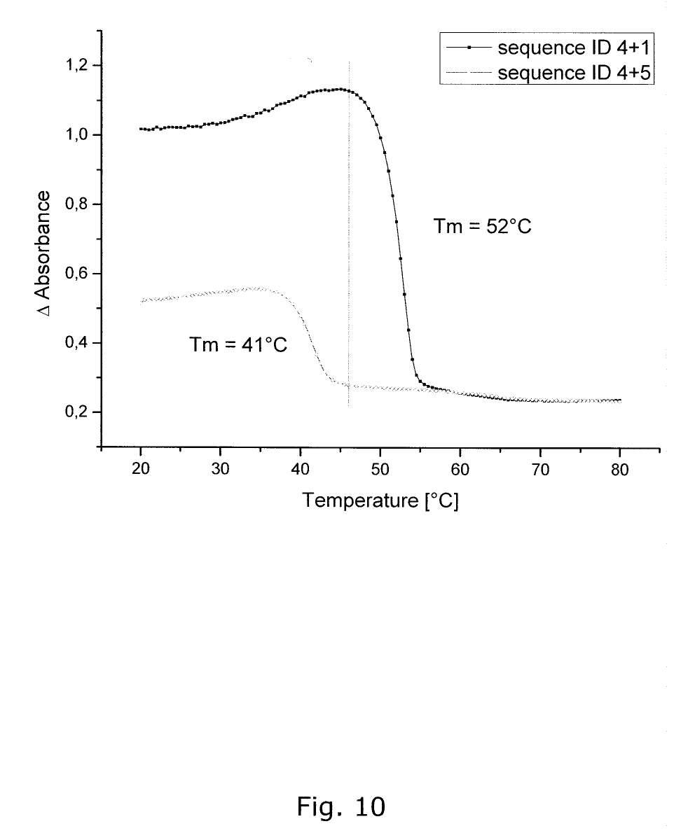

Thermal denaturation curves recorded in a UV experiment at 260 nm wavelength showing hybridisation of sequence ID4 and ID1 compared to the hybridisation of sequence ID4 and ID5 at 0.125 μM DNA concentration of each single stranded DNA sequence (FIG. 10).

### Example 5

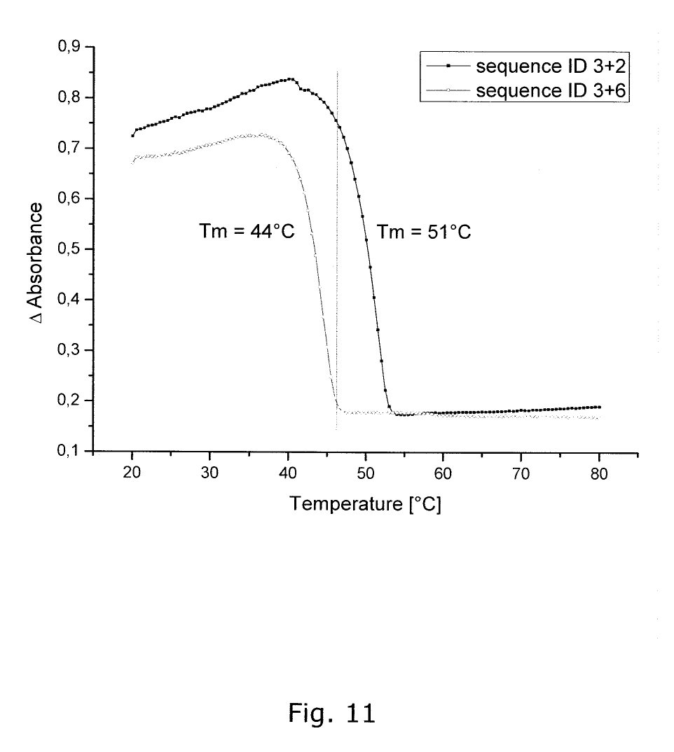

Thermal denaturation curves recorded in a UV experiment at 260 nm wavelength showing hybridisation of sequence ID3 and ID2 compared to the hybridisation of sequence ID3 and ID6 at 0.125 μM DNA concentration of each single stranded DNA sequence (FIG. 11)

### Example 6

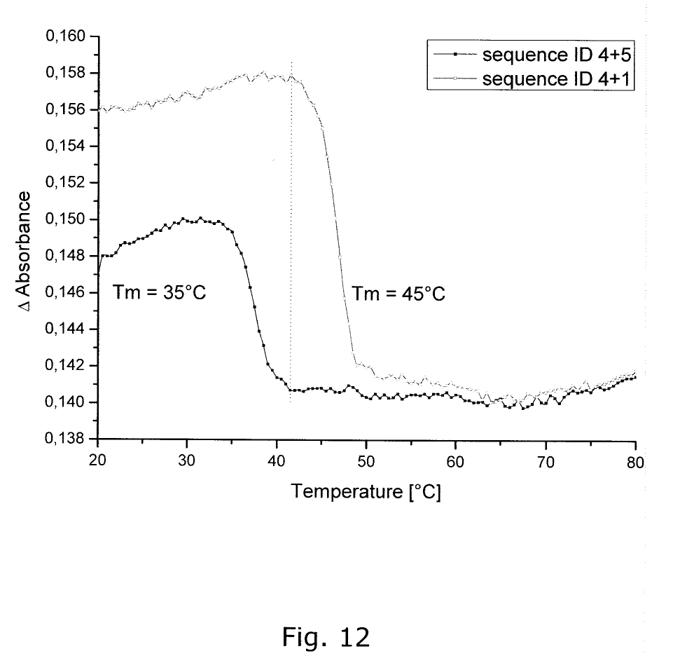

Thermal denaturation curves recorded in a UV experiment at 260 nm wavelength showing hybridisation of sequence ID4 and ID1 compared to the hybridisation of sequence ID4 and ID5 at 12.5 nM DNA concentration of each single stranded DNA sequence (FIG. 12).

### Example 7

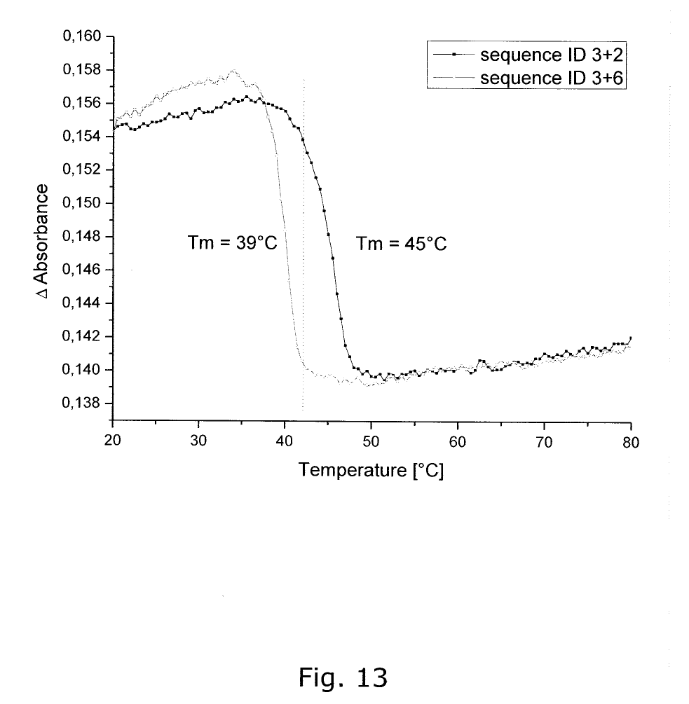

Thermal denaturation curves recorded in a UV experiment at 260 nm wavelength showing hybridisation of sequence ID3 and ID2 compared to the hybridisation of sequence ID3 and ID6 at 0.125 μM DNA concentration of each single stranded DNA sequence (FIG. 13).

### Example 8

### Membrane Anchoring Moiety is Required for Assembly of Lipid Membranes

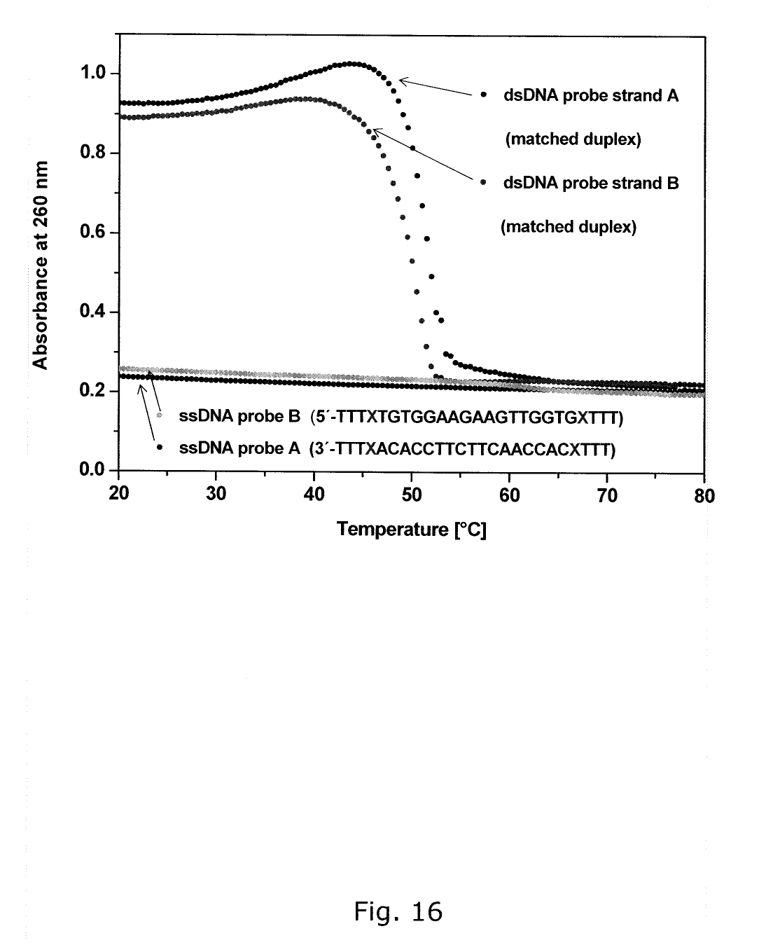

In this experiment, the effect of adding a target oligonucleotide was tested as determined by liposome assembly. As shown in FIG. 16, lipid assembly only takes when the target oligonucleotide is added.

The experiment was repeated in the presence of serum and yeast cell extract, and neither did affect liposome assembly. Thus, liposome assembly is a robust process that can take place in complex solutions.

### Example 9

### Mismatch Discrimination Studies

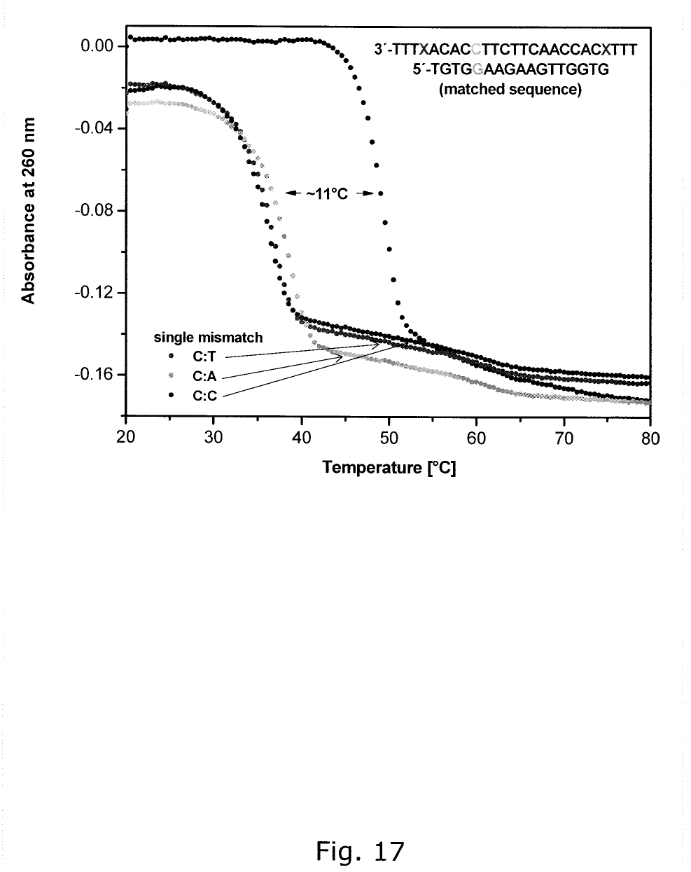

In this experiment, the effect of mismatches was analysed (FIG. 17). Thus, a perfectly matched target oligonucleotide was compared to three sequences with mismatches. A mismatch decreased the tm value with about 11° C., seen in the 11° C. shift of the temperature for liposome assembly. The shift in temperature was similar to temperature shift seen for unmodified oligonucleotides.

TABLE 1
Mismatch discrimination data

| | | | | | |
|---|---|---|---|---|---|
| | | Tₘ | ΔTₘ | Tₘ | ΔTₘ |
| Nr. | Duplex | [° C.] | [° C.] | [° C.] | [° C.] |

| | | | |
|---|---|---|---|
| target | 3′-TTTXACACCTTCTTCAACCACXTTT | with liposomes^{c} | without liposomes^{d} |

| | | | | | |
|---|---|---|---|---|---|
| 1 | 5′-TGTGGAAGAAGTTGGTG | 48 | — | 47 | — |
| 2 | 5′-TGTGTAAGAAGTTGGTG | 36 | −12 | 37 | −10 |
| 3 | 5′-TGTGAAAGAAGTTGGTG | 38 | −10 | 38 | −11 |
| 4 | 5′-TGTGCAAGAAGTTGGTG | 36 | −12 | 37 | −11 |

[a] X denotes the polyaza crown ether base surrogate. [b] Conditions: Tm values/° C. (ΔTm = change in Tm value calculated relative to the DNA: DNA reference duplex measured as the maximum of the first derivative of the melting curve (A₂₆₀ vs. temperature) recorded in medium salt buffer (10 mM HEPES, 110 mM Na+, pH 7.0). ^{[c]} 62 nM concentrations of the two complementary strands in presence of liposomes ^{[d]} 1 μM concentrations of the two complementary strands in absence of liposomes. Exp. error: ±1° C.

### Example 10

### Detection of an Anthrax Sequence

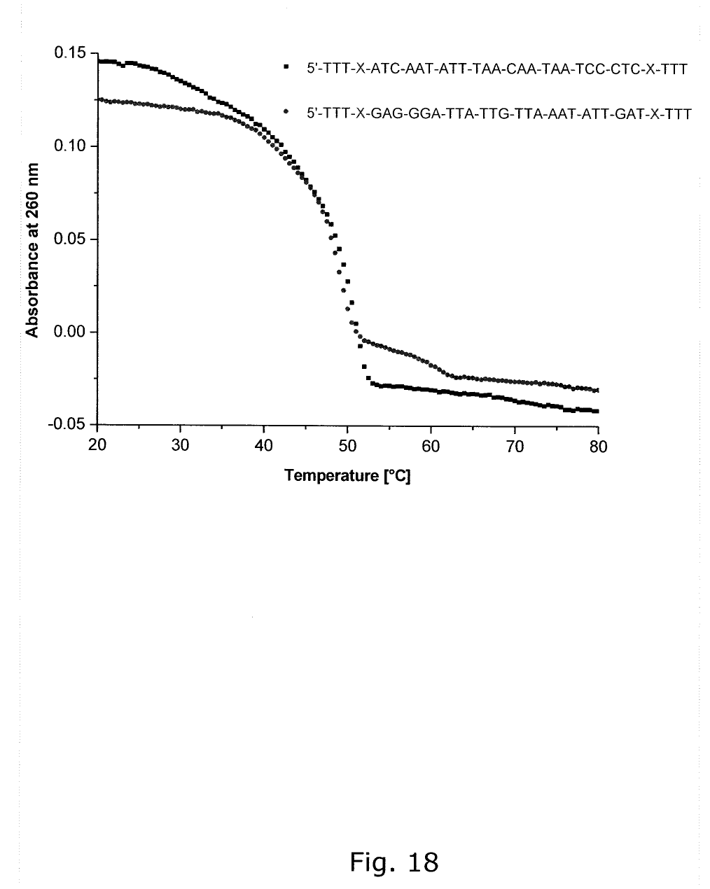

In this experiment, lipid assembly was analysed for a target sequence of Anthrax bacteria. As seen in FIG. 18, lipid assembly also took place with an Anthrax sequence.

### Example 11

### Detection of Sequences from Staphylococcus aureus

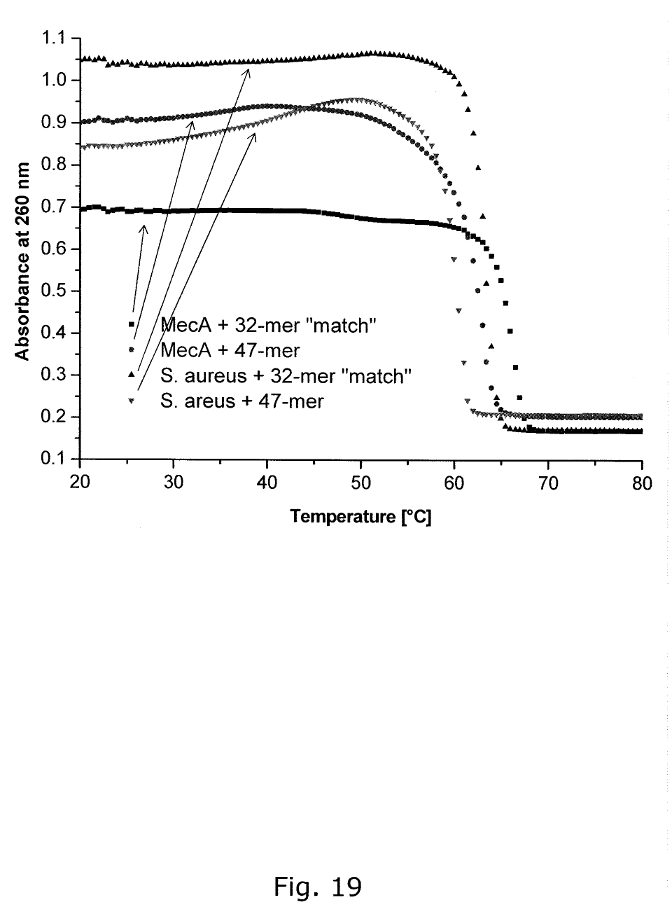

In this experiment, lipid assembly was analysed for a target sequence of Staphylococcus aureus bacteria. As seen in FIG. 19 lipid assembly also took place with an Anthrax sequence.

### Example 12

### Detection of Longer Fragments

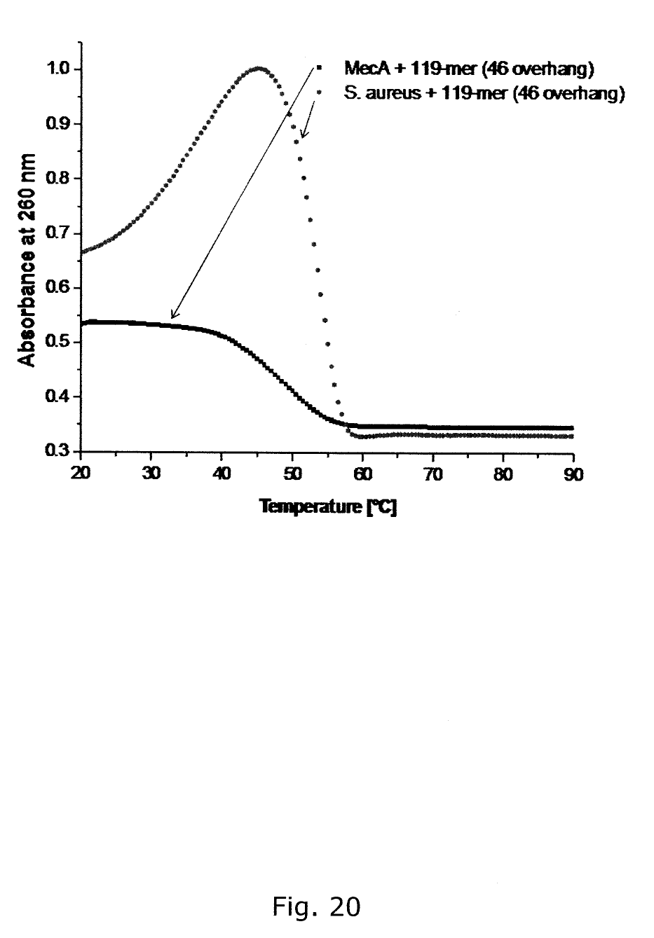

In this experiment, lipid assembly was analysed for a target sequence of S. aureus (FIG. 20). As an extra feature, the target oligonucleotide was rather long, i.e. 119 nucleotides. Again liposome assembly took place. Thus, the method can also be employed for target fragments that are substantially longer than the amphiphilic oligonucleotide probe.

### Example 13

### 3-strand design

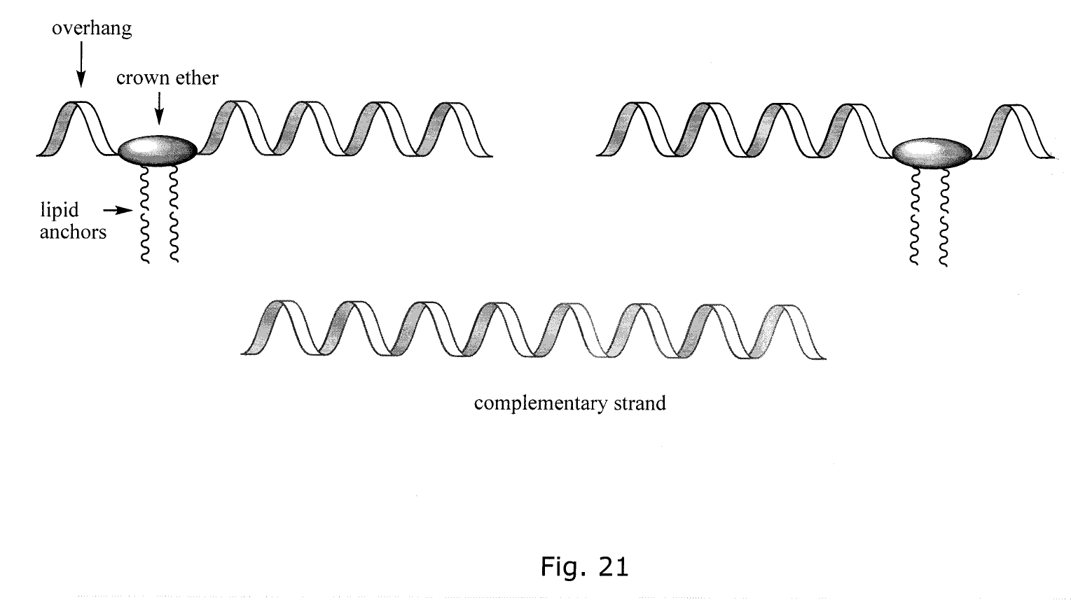

In this experiment, the amphiphilic oligonucleotide was represented by two oligonucleotides, each with complementary to the target oligonucleotide and each with a membrane anchor (FIG. 20). As shown in FIG. 21, the use of the two aforementioned oligonucleotides also mediated liposome assembly.

### Example 14

### Solid Supports

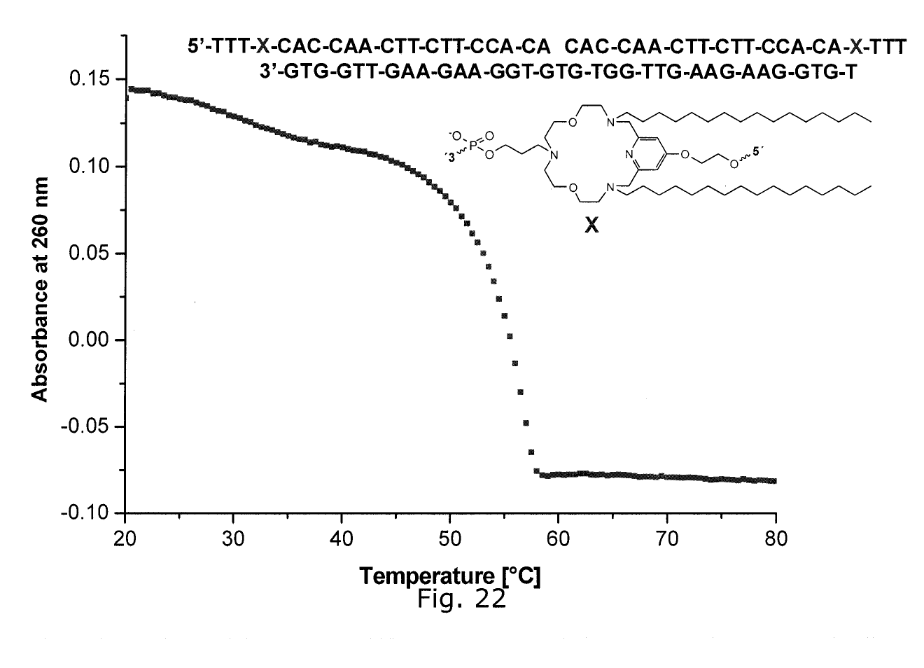

This example demonstrates immobilization of oligonucleotides of the invention to a supported membrane (FIG. 22). When a target oligonucleotide is added, liposomes where tethered to the surface within a time of 1-2 hours. About 90% assembly occurs within the first 15 minutes.

1. A method for constructing oligonucleotide-lipid architectures comprising:
a. Providing one or more amphiphilic oligonucleotides, which alone or together comprise at least two membrane anchors;
b. Providing a lipid membrane; and
c. Incubating the one or more amphiphilic oligonucleotides with the lipid membrane, under conditions allowing insertion of the membrane anchors into the lipid membrane.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. The method of claim 1, further comprising incubating, under conditions of hybridisation, the one or more amphiphilic oligonucleotides and the lipid membrane with a nucleic acid that comprises a region of complementarity to the one or more amphiphilic oligonucleotide.

35. The method of claim 1, wherein only one amphiphilic oligonucleotide is provided.

36. The method of claim 1, wherein the amphiphilic oligonucleotide comprises only two anchors and the two anchors of the amphiphilic oligonucleotide are separated by at least 5 nucleotides.

37. The method of claim 1, wherein the amphiphilic oligonucleotide comprises monomers selected from the group consisting of DNA, RNA, LNA, PNA, and morpholino.

38. The method of claim 1, wherein the length of the amphiphilic oligonucleotide is less than 35 nucleotides.

39. The method of claim 1, wherein the region of complementarity has a length of at least 10 nucleotides.

40. The method of claim 1, wherein the region of complementarity hybridised to a complementary RNA oligonucleotide has a melting temperature of at least 30° C.

41. The method of claim 1, wherein the membrane anchors are lipophilic moieties comprising an alkyl chain with a length selected from the group consisting of: at least 4 C atoms, at least 5 C-atoms, at least 6 C-atoms, at least 7 C-atoms, at least 8 C-atoms, at least 9 C-atoms, at least 10 C-atoms, at least 11 C-atoms, at least 12 C-atoms, at least 13 C-atoms, at least 14 C-atoms, at least 15 C-atoms, at least 16 C-atoms, at least 17 C-atoms, at least 18 C-atoms, at least 19 C-atoms and at least 20 C-atoms, or an alkenyl chain with a length selected from the group consisting of: at least 4 C atoms, at least 5 C-atoms, at least 6 C-atoms, at least 7 C-atoms, at least 8 C-atoms, at least 9 C-atoms, at least 10 C-atoms, at least 11 C-atoms, at least 12 C-atoms, at least 13 C-atoms, at least 14 C-atoms, at least 15 C-atoms, at least 16 C-atoms, at least 17 C-atoms, at least 18 C-atoms, at least 19 C-atoms and at least 20 C-atoms, or an alkynyl chain with a length selected from the group consisting of: at least 4 C atoms, at least 5 C-atoms, at least 6 C-atoms, at least 7 C-atoms, at least 8 C-atoms, at least 9 C-atoms, at least 10 C-atoms, at least 11 C-atoms, at least 12 C-atoms, at least 13 C-atoms, at least 14 C-atoms, at least 15 C-atoms, at least 16 C-atoms, at least 17 C-atoms, at least 18 C-atoms, at least 19 C-atoms and at least 20 C-atoms.

42. The method of claim 1, wherein the membrane anchors are lipophilic moieties that comprise one or more aromatic rings.

43. The method of claim 1, wherein the lipophilic moiety comprises a fatty acid selected from the group consisting of: butanoic acid; hexanoic acid; octanoic acid; decanoic acid; dodecanoic acid; tetradecanoic acid; hexadecanoic acid; 9-hexadecenoic acid; octadecanoic acid; 9-octadecenoic acid; 11-octadecenoic acid; 9,12-octadecadienoic acid; 9,12,15-octadecatrienoic acid; 6,9,12-octadecatrienoic acid; eicosanoic acid; 9-eicosenoic acid; 5,8,11,14-eicosatetraenoic acid; 5,8,11,14,17-eicosapentaenoic acid; docosanoic acid; 13-docosenoic acid; 4,7,10,13,16,19-docosahexaenoic acid and tetracosanoic acid.

44. The method of claim 1, wherein at least one membrane anchor comprises a polyaza crown ether with two lipophilic substituents.

45. The method of claim 1, wherein a first membrane anchor is located at least 1 nucleotide from the 5′ end of the amphiphilic oligonucleotide and wherein a second membrane anchor is located at least 1 nucleotides from the 3′ end of the amphiphilic oligonucleotide.

46. The method of claim 1, wherein the lipid membrane is part of a vesicle, a micelle, a cell membrane or a cellular membrane.

47. The method of claim 46, wherein the size of the vesicle or micelle is selected from the group consisting of: more than 20 nm, more than 50 nm, more than 75 nm, more than 100 nm, more than 150 nm and more than 200 nm.

48. The method of claim 46, wherein the size of the vesicle or micelle is selected from the group consisting of between 20 and 300 nm, between 50 and 200 nm and between 50 and 100 nm.

49. The method of claim 46, wherein the vesicle or micelle comprises a detectable marker selected from the group consisting of: a fluorescent group, a quantum dot and a metal nanoparticle.

50. The method of claim 49, wherein the detectable marker resides within the vesicle or micelle.

51. The method of claim 1, wherein the nucleic acid that comprises a region of complementarity to the amphiphilic oligonucleotide, is derived from a biological material selected from the group consisting of: a mRNA, a microRNA, a rRNA, a tRNA, a chromosomal DNA, and a PCR product.

52. The method of claim 1, further comprising detecting the presence of a target nucleic acid.

53. The method of claim 1, further comprising quantifying the presence of a target nucleic acid.

54. The method of claims 52, wherein the detection comprises measuring optical density.

55. The method of claim 34, further comprising separating the nucleic acid that comprises a region of complementarity to the amphiphilic oligonucleotide from other nucleic acids.

56. The method of claim 34, wherein the nucleic acid that comprises a region of complementarity to the amphiphilic oligonucleotide is an oligonucleotide capable of immobilisation on a solid support.

57. The method of claim 1, wherein the amphiphilic oligonucleotide is capable of immobilisation on a solid support.

58. The method of claim 57, wherein the solid support is a microarray.

59. An amphiphilic oligonucleotide comprising at least two membrane anchors, wherein said oligonucleotide is configured to allow insertion of the membrane anchors into a lipid membrane.

60. A lipid membrane comprising the oligonucleotide described in claim 59.

61. An oligonucleotide-lipid architecture comprising an oligonucleotide that comprises at least two membrane anchors joined to a lipid membrane.

62. The method of claim 1, further comprising formulating said oligonucleotide-lipid architecture into a pharmaceutical.

63. A pharmaceutical comprising the amphiphilic oligonucleotide of claim 59.

64. A method of administering an amphiphilic oligonucleotide to a person in need thereof comprising: providing the amphiphilic oligonucleotide of claim 59 and administering said amphiphilic oligonucleotide to a person in need thereof.

65. A kit for detecting the presence of a nucleic acid, the kit comprising:
an amphiphilic oligonucleotide as described in claim 59, a lipid membrane as described in claim 60, or an oligonucleotide-lipid architecture as described in claim 61 and instructions for detecting the presence of a nucleic acid.


SEQUENCE LISTING (11 sequences)

SEQ ID NO: 1 - DNA, 17 bp, Artificial Sequence
  Synthetic oligonucleotide
tgtggaagaagttggtg

SEQ ID NO: 2 - DNA, 17 bp, Artificial Sequence
  Synthetic oligonucleotide
caccaacttcttccaca

SEQ ID NO: 3 - DNA, 17 bp, Artificial Sequence
  Synthetic oligonucleotide
tgtgtaagaagttggtg

SEQ ID NO: 4 - DNA, 17 bp, Artificial Sequence
  Synthetic oligonucleotide
cacctacttcttccaca

SEQ ID NO: 5 - DNA, 23 bp, Artificial Sequence
  Synthetic oligonucleotide
ttttgtggaagaagttggtgttt

SEQ ID NO: 6 - DNA, 23 bp, Artificial Sequence
  Synthetic oligonucleotide
tttcaccaacttcttccacattt

SEQ ID NO: 7 - DNA, 30 bp, Artificial Sequence
  Synthetic oligonucleotide
tttatcaatatttaacaataatccctcttt

SEQ ID NO: 8 - DNA, 30 bp, Artificial Sequence
  Synthetic oligonucleotide
tttgagggattattgttaaatattgatttt

SEQ ID NO: 9 - DNA, 20 bp, Artificial Sequence
  Synthetic oligonucleotide
tttcaccaacttcttccaca

SEQ ID NO: 10 - DNA, 20 bp, Artificial Sequence
  Synthetic oligonucleotide
caccaacttcttccacattt

SEQ ID NO: 11 - DNA, 34 bp, Artificial Sequence
  Synthetic oligonucleotide
tgtggaagaagttggtgtgtggaagaagttggtg